(19) United States
(12) Patent Application Publication
(10) Pub. No.: US 2010/0204556 A1
(43) Pub. Date: Aug. 12, 2010

(54) PHYSIOLOGICAL PARAMETER SENSORS

(75) Inventors: Richard Blakley, Livermore, CA (US); John Mackay, San Jose, CA (US)

Correspondence Address: KNOBBE MARTENS OLSON & BEAR LLP, 2040 MAIN STREET, FOURTEENTH FLOOR, IRVINE, CA, 92614, US

(73) Assignee: KEIMAR, INC., Pleasanton, CA (US)

(21) Appl. No.: 12/704,408

(22) Filed: Feb. 11, 2010

Related U.S. Application Data
(60) Provisional application No. 61/152,183, filed on Feb. 12, 2009.

(51) Int. Cl. A61B 5/145

(52) U.S. Cl. 600/353

(57) ABSTRACT

A blood gas concentration sensor includes a first housing at least partially defining a first chamber containing a first electrolyte, a first electrode in the first chamber, and a second electrode in the first chamber and substantially parallel to the first electrode. The first housing includes a gas permeable material. The first electrode includes sides and an end. The sides of the first electrode are surrounded by a first insulating layer. The end of the first electrode is in contact with the first electrolyte. The end of the first electrode is not in contact with the first housing. The first electrode includes a first metal. The second electrode includes a second metal. A potential difference between the first metal and the second metal is at least about 0.5 volts.

### CROSS-REFERENCE TO RELATED APPLICATIONS

This application claims priority benefit of U.S. Provisional Patent Application No. 61/152,183, filed Feb. 12, 2009, which is incorporated herein by reference in its entirety.

### BACKGROUND

1. Field

The present application generally relates to probes and sensors for measuring physiological parameters, and more particularly relates to implantable probes and sensors for ascertaining parameters of body fluids such as temperature, gas concentrations, pH, and pressure.

2. Description of Related Technology

Determination of cardiac output, arterial blood gases, blood pressure, and other hemodynamic or cardiovascular parameters is critically important in the treatment and care of patients, particularly those undergoing surgery or other complicated medical procedures and those under intensive care. Such parameters can provide important patient status information to caregivers that can inform treatment decisions.

Typically, cardiac output measurements are made using pulmonary artery thermodilution catheters, which can have inaccuracies of 20% or greater. The use of such thermodilution catheters increases hospital costs while exposing the patient to potential infectious, arrhythmogenic, mechanical, and therapeutic misadventure. Blood gas measurements have been commonly made by removing a blood sample from the patient and transporting the sample to a lab for analysis. The caregiver must wait for the results to be reported by the lab, a delay of 20 minutes being typical and longer waits not being unusual.

“Point-of-care” blood testing systems allow blood sample analysis at a patient's bedside or in the area where the patient is located. Such systems include portable and handheld units and modular units that fit into a bedside monitor and can determine parameters such as metabolite and blood gas concentrations. While most point-of-care systems require the removal of blood from the patient for bedside analysis, a few do not. In some systems, intermittent blood gas and metabolite measurements are made by drawing a sufficiently large blood sample into an arterial line to ensure an undiluted sample at a sensor located in the line. After analysis, the blood is returned to the patient, the line is flushed, and results appear on the bedside monitor. In other systems, such as those that measure the concentration of single or multiple metabolites in a patient's blood, blood is drawn into a syringe and placed into a vial or ampule, microfuged to separate plasma from platelets, and pipetted into a sample vial that is placed into a bench-top or floor-model analyzer for measurement. Such analyzers require many operating steps, are expensive and bulky and not readily accessible, practical, or affordable in many situations and settings.

A non-invasive technology, pulse oximetry, is available for estimating the percentage of hemoglobin in arterial blood that is saturated with oxygen. Although pulse oximeters are capable of estimating arterial blood oxygen content, they are not capable of measuring parameters such as carbon dioxide content, pH, the partial pressure of oxygen, or venous oxygen content. Furthermore, pulse oximetry is commonly performed at the fingertip and can be skewed by peripheral vasoconstriction or even nail polish. Although pulse oximetry can also be used to measure blood metabolite concentrations, such measurements are generally not as precise and reliable as electrochemical measurements.

Blood pressure can be measured non-invasively using a blood pressure manometer connected to an inflatable cuff. This is the most common method outside of the intensive care environment. In critical care settings, at least 60% of patients have arterial lines. An arterial line consists of a plastic or solid polymer cannula inserted into a peripheral artery (commonly the radial or the femoral). The cannula is kept open and patent because it is connected to a pressurized bag of heparinized fluid such as normal saline. An external gauge also connects to the arterial cannula to reflect the column of fluid pressure in the artery. This system consists of an arterial line connected to a pressure transducer by saline-filled, non-compressible tubing. This converts the pressure waveform into an electrical signal displayed on the bedside monitor. The pressurized saline for flushing is provided by a pressure bag. Several potential sources of error exist in this system. First, any one of the many components in the system can fail. Second, the transducer position is critical because the pressure displayed is pressure relative to position of transducer. Thus, in order to accurately reflect blood pressure, the transducer should be at the level of the heart. Over-reading may occur if the transducer is placed too low, and under-reading may occur if the transducer is placed too high, relative to the heart. Third, the transducer must be zeroed to the atmospheric pressure at the time of measurement, otherwise the blood pressure will be incorrectly measured. Fourth, it is critical to have appropriate damping in the system. Inadequate damping will result in excessive resonance in the system, which causes an overestimate of systolic pressure and an underestimate of diastolic pressure. An under-damped trace is often characterized by a high initial spike in the waveform. The opposite occurs with over-damping. In both cases, the mean arterial pressure value is the accurate enough for clinical use.

Closed-loop systems provide a platform for directing treatment based on feedback from sensors such as those specifically described in the present disclosure. The most effective treatment generally occurs when the device can be continually adjusted in response to changing patient conditions. Unfortunately, none of the available systems or methods for blood gas analysis provides for a reliable, closed-loop system having accurate, direct, and continuous in vivo measurements of arterial and venous oxygen partial pressure, carbon-dioxide partial pressure, pH, and temperature while presenting minimal risk to the patient.

### SUMMARY

For purposes of summarizing the invention and the advantages achieved over the prior art, certain objects and advantages of the invention are described herein. Of course, it is to be understood that not necessarily all such objects or advantages need to be achieved in accordance with any particular embodiment. Thus, for example, those skilled in the art will recognize that the invention may be embodied or carried out in a manner that achieves or optimizes one advantage or group of advantages as taught or suggested herein without necessarily achieving other objects or advantages as may be taught or suggested herein.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

An intravascular sensor assembly or probe is described herein that comprises sensors for measuring, simultaneously and continuously, one or more, and preferably, three or more, characteristics of the blood flow of a human or animal. The sensors described herein include sensors for measuring blood temperature, pressure, pH, partial pressure of oxygen, and partial pressure of carbon dioxide. Other sensors, such as those for glucose, potassium, and other characteristics of the blood could be added or substituted. The probe is at least partially insertable into a vein or artery of a human or an animal, and comprises electronics that serve to condition, digitize, acquire, analyze, and display the signals of the sensors in the probe. The electronics may be housed at any place along the length of the probe, including but not limited to, the portion of the probe that is external to the vein or artery.

In certain embodiments, an implantable sensor for measuring blood temperature comprises a substantially uniformly doped silicon substrate comprising a first surface, an insulating layer over the first surface, a first contact over the insulating layer and proximate to the first side of the substrate, a first via through the insulating layer and electrically connecting the first contact and the substrate, a first barrier metal, a second contact over the insulating layer and proximate to a second side of the substrate, a second via through the insulating layer and electrically connecting the second contact and the substrate, and a second barrier metal layer. The first contact comprises a first metal comprising aluminum, copper, nickel, platinum, gold, or silver. The first via comprises the first metal. The first barrier metal layer is between the first via and the substrate, between the first via and the insulating layer, and between the first contact and the insulating layer. The first barrier metal layer comprises molybdenum, tungsten, titanium, or tantalum. The second side is opposite the first side. The second contact is spaced from the first contact. The second contact comprises the first metal. The second via comprises the first metal. The second via is spaced from the first via by a distance. The second barrier metal layer is between the second via and the substrate, between the second via and the insulating layer, and between the second contact and the insulating layer. The second barrier metal layer comprises molybdenum, tungsten, titanium, or tantalum. Upon application of a voltage between the first contact and the second contact, a measurable current propagates through a substantial portion of the substrate. Resistance of the substrate to the current is substantially linearly proportional to a temperature of the substrate between about 33° C. and about 41° C.

In certain embodiments, a temperature sensor comprises a substantially uniform substrate comprising a first material and comprising a first surface, a first contact over the first surface and proximate to a first side of the substrate, and a second contact over the first surface and proximate to a second side of the substrate. The second side is opposite the first side. The second contact spaced from the first contact by a first distance. The first contact comprises a second material different from the first material. The second contact comprises the second material. Upon application of a voltage between the first contact and the second contact, a measurable current propagates through a substantial portion of the substrate.

In certain embodiments, a method of manufacturing a temperature sensor comprises forming a first contact over a first surface of a substantially uniform substrate and proximate to a first side of the substrate and forming a second contact over the first surface of the substrate and proximate to a second side of the substrate. The second side is opposite the first side. After forming the first contact and the second contact and upon application of a voltage between the first contact and the second contact, a measurable current propagates through a substantial portion of the substrate.

In certain embodiments, a method of ascertaining temperature comprises applying a voltage between a first contact and a second contact. The first contact is over a first surface of a substantially uniform substrate and proximate to a first side of the substrate. The second contact is over the first surface of the substrate and proximate to a second side of the substrate. The second side is opposite the first side. The method further comprises measuring a current propagating through a substantial portion of the substrate and determining temperature at least partially based on the measured current.

In certain embodiments, an implantable galvanometric sensor for measuring blood gas concentration comprises a first gas permeable tube at least partially defining a first chamber containing a first electrolyte, a second gas permeable tube at least partially in the first chamber and at least partially defining a second chamber containing a second electrolyte, a first sensing electrode extending into the second chamber from a first direction, a third tube at least partially in the second chamber, and a first reference electrode in the third chamber and extending into the second chamber from the first direction. The first sensing electrode comprises a first insulated wire having an exposed end in contact with the second electrolyte and not in contact with the second gas permeable tube. The exposed end of the first insulated wire is a substantially radial cross-section of the first insulated wire. The first sensing electrode comprises a first metal. The third tube comprises sides and an end comprising a first frit. The sides of the third tube are gas impermeable. The sides and the end of the third tube at least partially defining a third chamber containing a third electrolyte. The first reference electrode is substantially parallel to the first sensing electrode. The first reference electrode comprises a second metal. A potential difference between the first metal and the second metal is at least about 0.5 volts.

In certain embodiments, a blood gas concentration sensor comprises a first housing at least partially defining a first chamber containing a first electrolyte, a first electrode in the first chamber, and a second electrode in the first chamber and substantially parallel to the first electrode. The first housing comprises a gas permeable material. The first electrode comprises sides and an end. The sides of the first electrode are surrounded by a first insulating layer. The end of the first electrode is in contact with the first electrolyte. The end of the first electrode is not in contact with the first housing. The first electrode comprises a first metal. The second electrode comprises a second metal. A potential difference between the first metal and the second metal is at least about 0.5 volts.

In certain embodiments, a blood gas concentration sensor comprises a first housing at least partially defining a first chamber containing a first electrolyte, a first wire comprising an exposed end comprising a first metal in contact with the first electrolyte, and a second wire comprising a second metal. The first housing comprises a gas permeable material. A potential difference between the first metal and the second metal is at least about 0.5 volts.

In certain embodiments, a galvanometric sensor comprises a plurality of electrodes suspended and separated slightly from each other in an electrolyte configured to support an electrochemical reaction. The electrodes and the electrolyte are at least partially suspended in a cell that is permeable to oxygen. The electrodes comprise electrochemically different materials. Upon suspension in the electrolyte, the electrodes generate a voltage that is monotonically dependent upon the oxygen concentration in the electrolyte.

In certain embodiments, a polarographic sensor comprises a plurality of electrodes suspended and separated slightly from each other in an electrolyte configured to support an electrochemical reaction. The electrodes and the electrolyte are at least partially contained in a cell that is permeable to oxygen. The electrodes comprise conductive materials that are substantially electrochemically identical. Upon suspension in the electrolyte and application of an appropriate voltage, the electrodes generate a current that is monotonically dependent upon the oxygen concentration in the electrolyte.

In certain embodiments, a probe for ascertaining parameters of blood in a vessel of a patient comprises a housing having an internal wall, a plurality of sensors in the housing, a barrier system between the sensors and in contact with the inner wall of the housing, and a conductor through the barrier system. Each sensor comprises an electrolyte. The barrier system is configured to physically and electrically isolate the sensors. The barrier system may comprise a material selected from the group consisting of butyl rubber, silicone rubber, soft durometer polymer, urethane, vinyl, rubber, and silicone gel. The barrier system may comprise at least one feature proximate to the inner wall of the housing. The at least one feature may be configured to form a wiper action on the inner wall of the housing. The at least one feature may comprise an air chamber. The at least one feature may comprise a chamber comprising an electrically insulating fluid. The electrically insulating fluid may comprise air. The housing may comprise an aperture at least partially covered by the barrier system. The barrier system may comprise an inner chamber, and the probe may further comprise a conduit in fluid communication with the inner chamber. The barrier system may be fused to the housing. The barrier system may comprise a first barrier, a second barrier, and a longitudinal gap between the first barrier and the second barrier. The longitudinal gap may comprise a material selected from the group consisting of compliant polymer, compliant monomer, oil, and gel. The housing may comprise a plurality of sealed longitudinal parts.

In certain embodiments, a method of manufacturing a probe comprising a plurality of sensors configured to ascertain parameters of blood in a vessel of a patient comprises inserting a barrier system molded around a substrate into a housing having an inner wall. The barrier system mechanically contacts the inner wall to form at least one chamber in the housing. The method further comprises at least partially filling the chamber with an electrolyte. At least partially filling the chamber may comprise adding the electrolyte through an aperture in the housing and the method may further comprise, after at least partially filling the chamber with the electrolyte, further inserting the barrier system into the housing. After further inserting, the barrier system at least partially covers the aperture. The method may further comprise, prior to at least partially filling the chamber, evacuating the chamber.

In certain embodiments, a method of manufacturing a probe comprising a plurality of sensors configured to ascertain parameters of blood in a vessel of a patient comprises molding a barrier system around a substrate and inserting the barrier system into a housing having an inner wall. The barrier system mechanically contacts the inner wall to form at least one chamber in the housing. The method further comprises at least partially filling the chamber with an electrolyte. At least partially filling the chamber may comprise adding the electrolyte through an aperture in the housing and the method may further comprise, after at least partially filling the chamber with the electrolyte, further inserting the barrier system into the housing. After further inserting, the barrier system at least partially covers the aperture. At least partially filling the chamber may comprise evacuating the chamber. Molding the barrier system may comprise forming at least one feature on an exterior surface of the barrier system. During inserting the barrier system, the at least one feature may act as a wiper on the inner wall of the housing. Inserting the barrier system may comprise at least partially filling the at least one feature with a fluid. The fluid may comprise air. The fluid may comprise oil. Molding the barrier system may comprise forming an inner chamber. Inserting the barrier system may comprise at least partially evacuating the inner chamber and the method may further comprise, before at least partially filling the chamber with the electrolyte, at least partially filling the inner chamber with a fluid. After at least partially filling the inner chamber with the fluid, the barrier system mechanically contacts the inner wall of the housing. At least partially filling the inner chamber may comprise pressurizing the inner chamber to an ambient pressure. The method may further comprise fusing the barrier system to the housing. Fusing the barrier system to the housing may comprise at least one of laser heating, ultrasonic heating, plasma heating, and hot coil heating. Molding the barrier system may comprise molding a first barrier comprising a first material, molding a second barrier comprising a second material adjacent to the first barrier, and molding a third barrier comprising the first material adjacent to the second barrier. The second material is different than the first material. The second material may comprise at least one of compliant polymer, compliant monomer, oil, and gel.

### BRIEF DESCRIPTION OF THE DRAWINGS

These and other features, aspects, and advantages of the present disclosure are described with reference to the drawings of certain embodiments, which are intended to illustrate certain embodiments and not to limit the invention.

FIG. 1A is an isometric view of an example embodiment of a system for ascertaining blood characteristics.

FIG. 1B is a partially cut away plan view of an example embodiment of a kit comprising a system for ascertaining blood characteristics.

FIG. 2 is a cutaway and partially cross-sectional view of an example embodiment of a connector portion of a probe or sensor assembly.

FIG. 3A is a cutaway and partially cross-sectional view of an example embodiment of a measurement portion of a probe or sensor assembly.

FIG. 3B is a cutaway and partially cross-sectional view of another example embodiment of a measurement portion of a probe or sensor assembly.

FIGS. 3C-3G are cutaway and partially cross-sectional views of example embodiments of barrier systems.

FIG. 4 is a cross-sectional view of an example embodiment of a temperature sensor.

FIG. 5A is a cross-sectional view of another example embodiment of a temperature sensor.

FIG. 5B is a cross-sectional view of another example embodiment of a temperature sensor.

FIG. 6A is a cutaway and cross-sectional view of a portion of yet another example embodiment of a temperature sensor.

FIG. 6B is a cutaway and cross-sectional view of a portion of still yet another example embodiment of a temperature sensor.

FIG. 6C is a cutaway and cross-sectional view of a portion of a further example embodiment of a temperature sensor.

FIG. 7A is a cross-sectional view of an example blood gas concentration sensor.

FIG. 7B is a cross-sectional view of the blood gas concentration sensor of FIG. 7A taken along the line 7B-7B.

FIG. 8 is a cross-sectional view of an example embodiment of a blood gas concentration sensor.

FIG. 9 is a cross-sectional view of another example embodiment of a blood gas concentration sensor.

FIG. 10A is a cross-sectional view of yet another example embodiment of a blood gas concentration sensor.

FIG. 10B is a cross-sectional view of still another example embodiment of a blood gas concentration sensor.

### DETAILED DESCRIPTION

Although certain embodiments and examples are described herein, those of skill in the art will appreciate that the invention extends beyond the specifically disclosed embodiments and/or uses and obvious modifications and equivalents thereof. Thus, it is intended that the scope of the disclosed invention should not be limited by any particular embodiment(s) described herein.

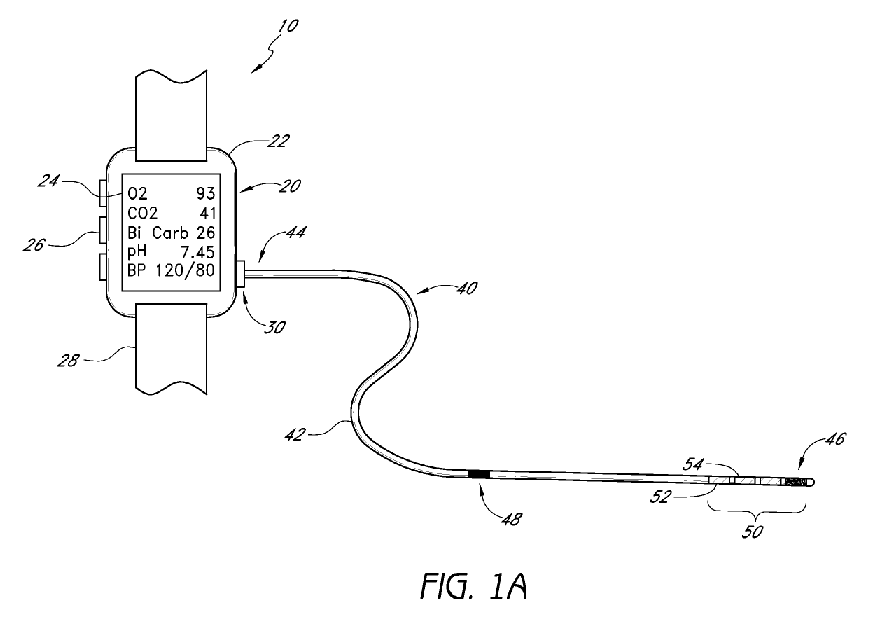

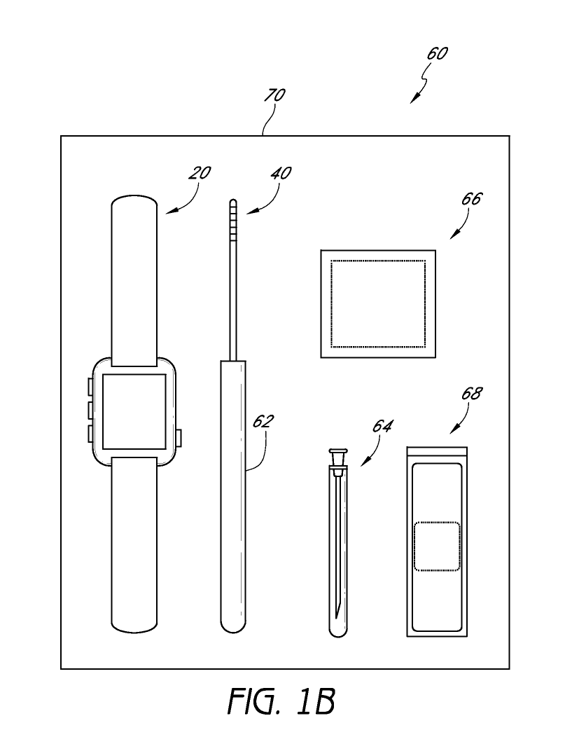

FIG. 1 illustrates an example embodiment of a system 10 for making intravascular measurements of physiological parameters or characteristics. The system 10 comprises a display module 20 and or more probes 40. As described in more detail herein, the display module 20 and the probe 40 are adapted for accurate and continuous in vivo measurement and display of body fluid parameters or characteristics such as partial pressure of oxygen (pO₂), partial pressure of carbon dioxide (pCO₂), pH, temperature, and pressure. In addition, cardiac output (CO) can be calculated by combining two pO₂ measurements obtained from a pair of probes 40, one disposed in an artery and the other in a vein. In certain such embodiments, each of the probes 40 may be connected to a single display module 20 or each of the probes 40 may be connected to a different display module 20. Alternatively or in addition to the aforementioned sensors, the probe 40 may include sensors for parameters such as potassium, sodium, calcium, bilirubin, hemoglobin/hematocrit, glucose, and lactate concentration and pressure. Additional features of example embodiments of the display module 20 and/or the probe 40 are described in U.S. patent application Ser. No. 12/552,081, filed Sep. 1, 2009, Ser. No. 12/172,181, filed Jul. 11, 2008, Ser. No. 12/027,933, filed Feb. 7, 2008, Ser. No. 12/027,915, filed Feb. 7, 2008, Ser. No. 12/027,905, filed Feb. 7, 2008, Ser. No. 12/027,902, filed Feb. 7, 2008, and Ser. No. 12/027,898, filed Feb. 7, 2008, and U.S. Pat. Nos. 6,616,614 and 7,630,747, the entire contents of each of which are incorporated herein by this reference as if set forth fully herein.

The display module 20 comprises a housing 22 (e.g., comprising plastic or polymer). In some embodiments, the display module 20 is sized so that the display module 20 can be worn on the patient or subject, for example on the patient's wrist, arm, or other limb. The display module 20 further comprises a display 24 (e.g., comprising a flat compact display based on electronic ink, liquid crystal, light emitting diode, combinations thereof, and the like) configured to present one or more ascertained parameters and/or other information. The display 24 is adapted to be readily visible to the attending medical professional or user. The display 24 may include backlighting or other features to enhance the visibility of the display 24.

In some embodiments, the display module further comprises an input device 26 (e.g., comprising buttons, keys, switches, trackball, touchscreen, etc.) to facilitate entry of instructions and/or viewing of data. In some embodiments, the display module 20 does not comprise an input device 26. In certain such embodiments, the display module 20 may automatically present different information on the display 24 at a rate consistent with medical practice. For example, each screen of the display 24 might appear for 3 seconds before being replaced by a subsequent screen. In certain such embodiments, the sequence of screens may be automatically chosen based on medical practice. In some embodiments, the display module 20 includes wireless communications capability configured to transmit physiologic parameters for viewing on a remote display, logging on a remote device, and/or to facilitate entry of patient parameters or other information into the display module 20 from a remote input device.

In some embodiments, the display module 20 comprises a band 28 that is coupled to the housing 22. The band 28 may be used to secure the display module 20 to the subject's wrist, arm, or to a location near the subject. If the subject is a newborn infant (neonate), the display module 20 may be strapped to the subject's torso. Other locations are also possible. In some embodiments, the band 28 comprises Velcro and/or elastic. In certain embodiments, the display module 20 comprises an adhesive or magnetic backing or a fastener (e.g., snap, hook, aperture, etc.) configured to attach the display module 20 to a location on or near the subject.

The display module 20 comprises electronic components configured to receive input from one or more probes 40 and to display information on the display 24. The electronic components may be configured for signal conditioning, collection, analog-digital conversion, analysis, and/or presentation. In some embodiments, the electronics components comprise voltage sources, current sources, operational amplifiers, passive electrical components, conductors, analog-digital converters, microprocessors, and/or other appropriate electronic components. In certain embodiments, the display module 20 comprises a processor, memory, and a bus system configured to provide communication between components of the display module 20. In some embodiments in which the display module 20 is part of a disposable kit, for example as described below, memory of the display module is pre-programmed with calibration values specific to the probe 40 of the kit. In some embodiments, the display module 20 comprises one or more display module connectors 30 for physical connection and communication with one or more probes 40. The display module connector 30 includes a receptacle adapted to receive, secure, and communicate with a corresponding connector on the proximal end of a probe 40. In some embodiments, the display module 20 comprises a wireless receiver (e.g., WiFi, RF, Bluetooth®, Zigbee®, and the like) for wireless connection and communication with one or more probes 40. Some of the components described herein may be located in different portions of a system (e.g., in a probe, in an intermediate electronics unit, etc.).

In some embodiments, the display module 20 comprises a power source (e.g., battery, solar panel) configured to provide power to the display module 20 for at least the expected lifetime of the probe 40. In some embodiments, the display module 20 is powered by being plugged into an outlet in a wall or another medical device. Combinations and variations thereof are also possible (e.g., solar panel and battery backup, rechargeable battery and outlet, power adapter, etc.).

FIG. 1B illustrates an example embodiment of a kit 60 comprising the system 10. The kit 60 comprises the display module 20 and one or more probes 40. In some embodiments, the display module 20 is low in cost so that it can be packaged together with one or a small plurality of probes 40. The kit 60 may optionally comprise additional accessories. For example, in the embodiment illustrated in FIG. 1B, the kit 60 comprises a probe holder 62, an introducer 66 (e.g., comprising a hypodermic needle), an alcohol swab 64, and a bandage 68. The kit 60 comprises a sterile container 70 (e.g., a sterilized plastic pouch) containing at least some of the components 20, 40, 62, 64, 66, 68 of the kit 60. In some embodiments, a kit comprises only some of the components illustrated in FIG. 1B. For example, a kit may comprise only the probe 40; the probe 40 and the probe holder 62; the probe 40, the probe holder 62, and the introducer 64; etc.

In some embodiments, the display module 20 is usable with multiple probes 40, either all simultaneously or sequentially. In certain such embodiments, the display module 20 comprises a handheld electronic device (e.g., Apple iPod Touch®, Dell Axim®, Hewlett Packard iPAQ®, smart phone, laptop computer, personal digital assistant, and the like). Certain such electronic devices comprise a processor, memory, bus system, battery, display, input device, wireless transmitter and/or receiver, and/or connector that may be adapted or programmed to communicate with one or more probes 40 and/or to present ascertained parameters. In certain such embodiments, the display module 20 may be sterilized and/or refurbished prior to reuse.

The probe 40 comprises a generally flexible elongate probe body or cannula or sleeve 42. The cannula or sleeve 42 may be formed of an insulating material, which provides strength and flexibility to the cannula 42. Examples of insulating materials include, but are not limited to, polymethylpentene, low density polyethylene, polytetrafluoroethylene, polypropylene, polycarbonate, polyimide, polyester, and nylon. In some embodiments, the insulating material is gas permeable over a portion or all of its length. The probe 40 has a proximal end or extremity 44 and a distal end or extremity 46, and may have a substantially uniform diameter over its entire length, or may have a variable diameter and variations of insulating materials to facilitate handling and/or robustness. In some embodiments, wall thickness of the cannula 42 in the sensor section 50 is between about 0.001 inches (approximately 25 micrometers (μm)) and about 0.003 inches (approximately 76 μm), for example about 0.0015 inches (approximately 38 μm). The probe 40 comprises a sensor section 50 at the distal end 46. The sensor section 50 may comprise one or more of the sensors described herein and/or other sensors (e.g., the sensors described in the applications incorporated herein by reference). In some embodiments, the probe 40 comprises a marker band 48, which may be used as a guide for the insertion of the probe 40 into the subject. In some embodiments, the marker band 48 is situated about 50 millimeters (mm) from the distal end 44 of the probe 40. In some embodiments, the marker band 48 is visible just outside the access point in the subject's skin when the probe 40 is inserted into the subject a desired amount. In some embodiments, the marker band comprises a radiopaque material and positioning may be guided by x-ray or other imaging techniques.

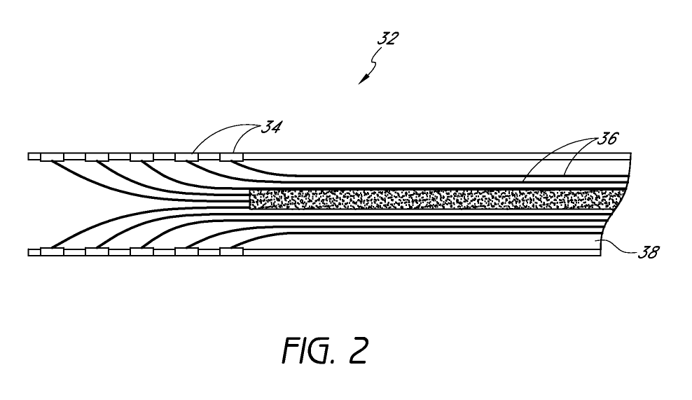

The cannula 42 is long enough so that when the distal end 46 is situated in a blood vessel, the proximal end 44 is accessible outside of the body and may be connected to and communicate with the display module 20. In certain embodiments, the proximal end 44 is configured to removably connect to and to communicate with display module 20 via a probe connector 32, for example as illustrated in FIG. 2. The probe connector 32 comprises a plurality of electrical contacts 34 configured to contact a corresponding plurality of electrical contacts on the display module connector 30. In some embodiments, the electrical contacts 34 are annularly or cylindrically disposed on the probe 40. Other types of bands or pads are also possible. For example, the electrical contacts 34 may be distributed on one or both sides of a flat connector such as a flex circuit. In some embodiments, the electrical contacts 34 provide a low-profile probe connector 32. The electrical contacts may comprise a conductive material such as, but not limited to, gold (Au), aluminum (Al), copper (Cu), platinum (Pt), silver (Ag), alloys thereof, combinations thereof, and the like.

In some embodiments, the cannula or sleeve 42 is cylindrical and comprises a material that is permeable or highly permeable to analyte gases, molecules, and/or ions. In certain such embodiments, the cannula 42 can form a large surface area circumferential window for one, multiple, or all of the sensors in the sensor section 50. A circumferential window may be advantageous by increasing or maximizing the permeable membrane area for a given sensor length. A circumferential window can also reduce or eliminate the “wall effect” artifact that may occur when a gas permeable membrane on the tip or one side of a probe 40 is partially or fully blocked from exposure to the blood when the probe is positioned against a vessel wall. Since the functionality of the sensors is at least partially affected by the ability of the target analyte in the blood to reach equilibrium with the solution in the chamber, even if the probe 40 is inadvertently placed against a vessel wall, the circumferential window can provide a gas permeation path into the sensor chambers so that equilibrium can be achieved.

In some embodiments, at least the portion of the cannula 42 comprises a surface treatment. In certain embodiments, the surface treatment is configured to inhibit adsorption of protein onto the outer surface of the cannula 42 and adhesion of blood components to the outer surface of the cannula 42 when disposed in the blood vessel of the patient. In certain embodiments, the surface treatment is configured to inhibit accumulation of thrombus, protein, or other blood components which might otherwise impair the blood flow in the vessel or impede the diffusion of target analyte into the sensors of the sensor section 50. In certain such embodiments, the surface treatment is configured to not significantly impede migration of carbon dioxide through the first gas permeable window and/or to not significantly impede migration of oxygen through the second gas permeable window.

The individual sensors of sensor section 50 each occupy a small longitudinal length of the probe 40. For example, in some embodiments, sensors of the sensor section 50 are each between about 5 mm and about 10 mm long (e.g., about 6 mm long). In some embodiments, the totality of the sensor section 50 is less than about 25 mm long. In certain embodiments, the length of the sensor section 50 is configured so that the distal end 46 of the probe 40 is small enough to be advanced through a tortuous vessel without significant impairment of either the vessel or the probe 40.

The probe 40 comprises a plurality of electrical conductors 36, which pass through the length of the cannula 42, through a bore or lumen 38, and attach to the plurality of electrical contacts 34. The electrical conductors 36 may comprise a conductive material such as, but not limited to, gold, aluminum, copper, platinum, silver, combinations thereof, and the like, covered by an insulating material, and are of substantially uniform diameter or thickness along their entire length. The electrical conductors 36 may be disposed on a flex circuit for a portion or all of their length within the probe 40. The electrical conductors 36 and electrical connectors 34 transmit electrical signals from the sensors in the sensor section 50 to the display module 20. In some embodiments, the electrical contacts 34 may be soldered, welded, or otherwise electrically coupled to the electrical conductors 36, which may be electrically coupled to the one or more sensors in the sensor section 50 of the probe 40. In some embodiments, distal ends of the electrical conductors 36 may form or be integrated with parts of the sensors.

In some embodiments, an implantable sensor assembly is configured to measure at least one of the following blood characteristics in a vein or an artery of a human or animal, simultaneously and continuously: oxygen concentration, carbon dioxide concentration, pH, temperature, and pressure.

Referring again to FIG. 1, the sensor section 50 of the probe 40 may comprise one or more gas permeable windows 52. In some embodiments, the cannula 42 may define the outer surface of the probe 40 and the substantial majority of the cannula 42 is filled with a flexible polymer such as ultraviolet-cured adhesive or adhesive encapsulant 54. The adhesive 54 may provide robustness to the cannula 42, anchor the electrical conductors 36 and/or sensors described herein, at least partially define chambers, and/or provide separation between chambers. In some embodiments, multiple types of adhesive 54 and/or other fillers may be utilized to improve performance and/or to make assembly of the probe 40 easier. For example, cyanoacrylate can be used for small-scale bonding and small gap filling, and an ultraviolet-cured adhesive 54 can be used for large gap filling and forming chamber walls. Other separators (e.g., insulating or chamber walls) are also possible. In some embodiments, all or a portion of the cannula 42 is gas permeable (e.g., permeable to oxygen and carbon dioxide) and is liquid and/or dissolved ion impermeable. In certain such embodiments, the cannula 42 comprises the gas permeable windows 52 (e.g., the portions of the cannula 42 between adhesive 54 is gas permeable).

The elements of the probe 40, including the connector 32, may be dimensioned to be passed through an inner bore of an introducer, such as a hypodermic needle, of a size suitable for accessing a blood vessel in the hand, wrist, or forearm. In some embodiments, the cannula 42 has an outer diameter between about 0.015 inches (approximately 380 μm) and about 0.030 inches (approximately 760 μm), for example about 0.020 inches (approximately 510 μm). In some embodiments, the cannula 42 has a cross-sectional area between about 0.00017 square inches (approximately 0.11 square millimeters (mm²)) and about 0.00071 square inches (approximately 0.45 mm²), for example about 0.00034 square inches (approximately 0.2 mm²). An example of an introducer for cannula 42 having a diameter of about 0.020 inches (approximately 510 μm) is a 20-gauge hypodermic needle having an inner diameter of at least 0.023 inches (approximately 584 μm). In some embodiments, the probe 40 has a length that allows the sensor section 50 to be inserted into a blood or other vessel in the hand, wrist, forearm, etc. while the connector 32 at the proximal end 44 of the probe 40 is physically connected to the display module 20. In certain such embodiments, the probe 40 has a length between about 20 centimeters (cm) and about 30 cm, for example about 25 cm.

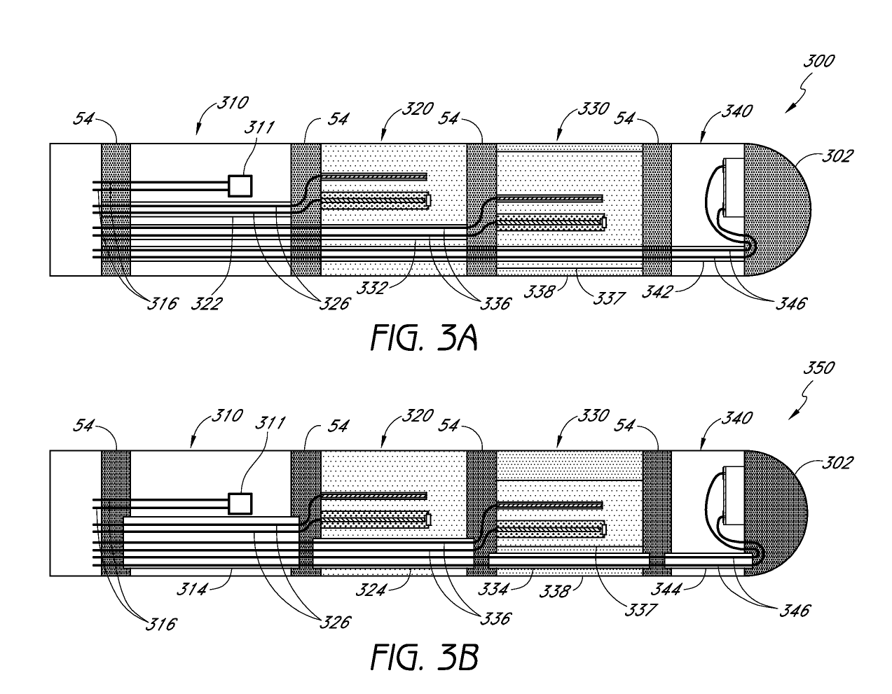

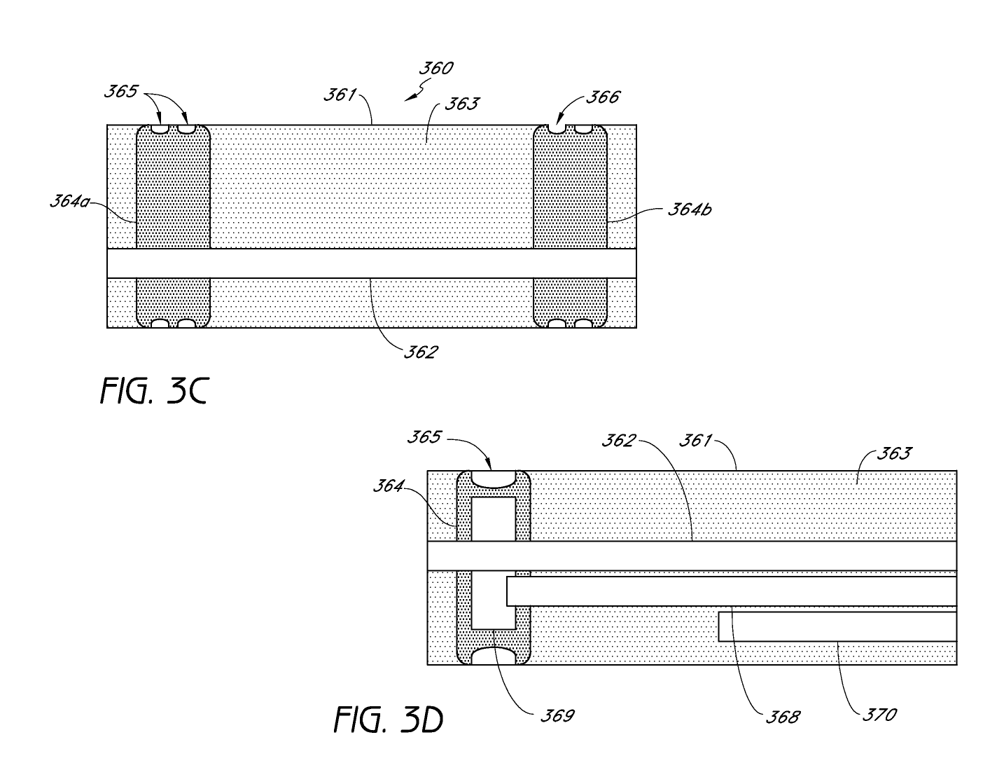

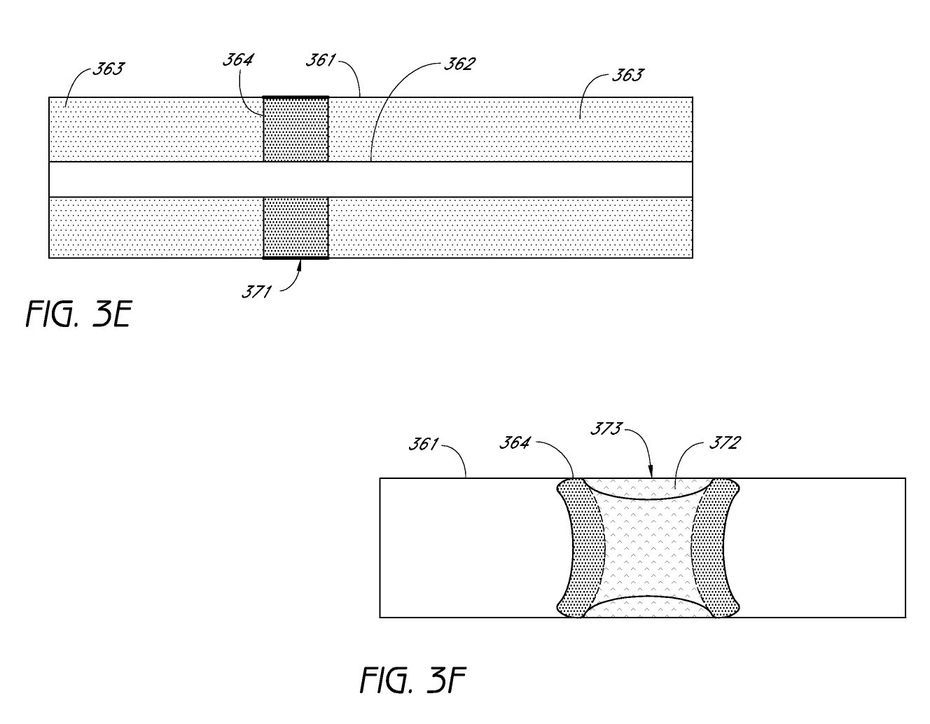

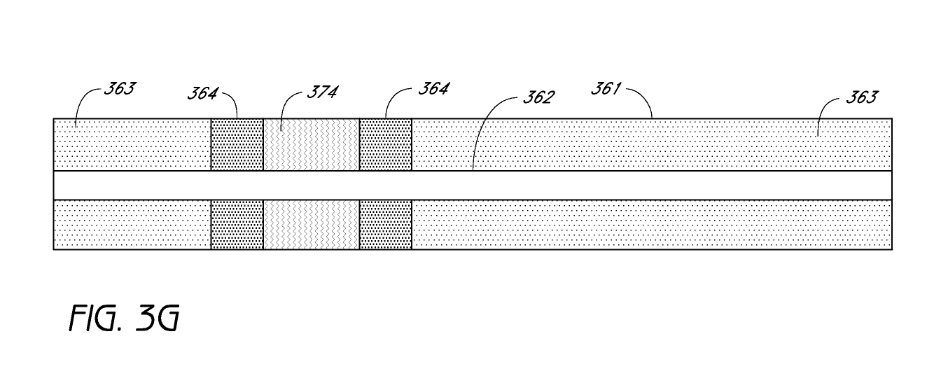

FIGS. 3A and 3B illustrate example embodiments of sensor sections 300 and 350, respectively, of a probe 40 that comprises a plurality of sensors 310, 320, 330, 340. The sensors 310, 320, 330, 340 are separated by barriers 54, each of which may comprise adhesive, oil, and/or solid polymer, for example as described herein. Additional sensors (e.g., proximal to the sensor 310) are also possible. The sensor section 300, 350 may comprise a tip 302, which may also comprise adhesive, oil, and/or solid polymer. The tip 302 may be porous to fluid or to specific ions in the fluid surrounding the sensor section 50. The tip 302 may be configured to allow safe routing of the probe 40 through a blood or other vessel of a subject. Other sensor separation means are also possible (e.g., discrete housings, membranes, etc.).

In some embodiments, the sensor 310 comprises a pH sensor or a pressure sensor, the sensor 320 comprises a carbon dioxide sensor distal to the pH sensor, the sensor 330 comprises an oxygen sensor distal to the carbon dioxide sensor, and the sensor 340 comprises a temperature sensor distal to the oxygen sensor. The sensor 310 comprises a black box 311 that is representative of other types of sensors that may be included in the probe 40, for example a pH sensor or a pressure sensor. In certain embodiments in which the sensor 310 comprises a pH sensor, the probe 40 also comprises a pressure sensor. In certain embodiments in which the sensor 310 comprises a pressure sensor, the probe 40 also comprises a pH sensor. Other types and arrangements of sensors are also possible. For example, the sensor section 50 may comprise additionally or alternatively comprise a pH sensor, a pressure sensor, an electrolyte concentration sensor, etc. For another example, the pH sensor could be between the oxygen sensor and the carbon dioxide sensor. For yet another example, the sensor section 300 could comprise one, two, three, four, or more sensors arranged in any desired order. In some embodiments, the probe comprises a pH sensor, a plurality of oxygen sensors, a carbon dioxide sensor, and a pressure sensor.

The sensor 340 is electrically connected to electrical contacts 34 via electrical conductors 346 (e.g., as illustrated in FIG. 2). In the embodiment illustrated in FIG. 3A, the electrical conductors 346 are routed through a conduit 342 extending from the tip 302, through the sensors 340, 330, 320, 310, to the proximal end of the sensor section 300. In other configurations, the conduit 342 may extend from proximal to the sensor 340, through the sensors 330, 320, 310, to the proximal end of the sensor section 300. In other configurations, the conduit 342 may extend from proximal to the sensor 340, through the sensors 330, 320, 310, to the proximal end of the sensor section 300. Although depicted in FIG. 3A as being substantially the same size, at least some of the conduits 342, 332, 322 may be different sizes. Although depicted in FIG. 3A as being spaced from each other, at least some of the conduits 342, 332, 322 may adjacent to each other. In some embodiments, connectors 316, 326, 336, 346 are disposed on a flex circuit that also provides support for some or all of the sensors in sensor section 50 and extends throughout most or all of the probe 40.

In the embodiment illustrated in FIG. 3B, the electrical conductors 346 are routed through a first conduit 344 extending from the tip 302, through the sensor 340, to the adhesive 54 between the sensor 340 and the sensor 330, through the adhesive 54 between the sensor 340 and the sensor 330, through a second conduit 334 extending from the adhesive 54 between the sensor 340 and the sensor 330, through the sensor 330, to the adhesive 54 between the sensor 330 and the sensor 320, through the adhesive 54 between the sensor 330 and the sensor 320, through a third conduit 324 extending from the adhesive 54 between the sensor 330 and the sensor 320, through the sensor 320, to the adhesive 54 between the sensor 320 and the sensor 310, through the adhesive 54 between the sensor 320 and the sensor 310, through a fourth conduit 314 extending from the adhesive 54 between the sensor 320 and the sensor 310, through the sensor 310, to the adhesive 54 proximal to the sensor 310. Although depicted in FIG. 3B as being different sizes for illustration purposes, at least some of the conduits 344, 334, 324, 314 may be substantially the same size. Other configurations are also possible. For example, the first conduit 344 may extend from the tip 302, through the sensor 340, through the adhesive 54 between the sensor 340 and the sensor 330, and through the sensor 330. For another example, the first conduit 344 may extend from the adhesive 54 between the sensor 340 and the sensor 330 and through the sensor 330. Although illustrated in FIGS. 3A and 3B as being proximate to a side of the sensor section 300, 350, the conduits may be proximate to the center of the sensor section 300, 350.

In some embodiments in which the sensor 330 comprises a first housing 337 and a second housing 338, the conduit 342 extends through the first housing 337 (e.g., as illustrated in FIG. 3A). In some embodiments in which the sensor 330 comprises a first housing 337 and a second housing 338, the conduit 334 extends between the first housing 337 and the second housing 338 (e.g., as illustrated in FIG. 3B). Other configurations are also possible. For example, the conduit 342 may extend between the first housing 337 and the second housing 338 and the conduit 334 may extend through the first housing 337.

The sensor 330 is electrically connected to electrical contacts 34 via electrical conductors 336 (e.g., as illustrated in FIG. 2). In the embodiment illustrated in FIG. 3A, the electrical conductors 336 are routed through a conduit 332 extending from the adhesive 54 between the sensor 320 and the sensor 330, through the sensors 320, 310, to the proximal end of the sensor section 300. In the embodiment illustrated in FIG. 3B, the electrical conductors 336 are routed through the adhesive 54 between the sensor 330 and the sensor 320, through the third conduit 324, through the adhesive 54 between the sensor 320 and the sensor 310, and through the fourth conduit 314.

The sensor 320 is electrically connected to electrical contacts 34 via electrical conductors 326 (e.g., as illustrated in FIG. 2). In the embodiment illustrated in FIG. 3A, the electrical conductors 326 are routed through a conduit 322 extending from the adhesive 54 between the sensor 320 and the sensor 310, through the sensor 310, to the proximal end of the sensor section 300. In the embodiment illustrated in FIG. 3B, the electrical conductors 326 are routed through the adhesive 54 between the sensor 320 and the sensor 310 and through the fourth conduit 314. Other combinations of conduits are also possible. For example, certain of the conduits may be coaxial with each other. Sensor sections 50, 300, 350 without conduits are also possible.

### Multiple Sensor Separation

In embodiments in which the probe 40 comprises a plurality of sensors configured to sense multiple parameters of blood, the sensors may be used interdependently and/or intradependently. In some embodiments, the electrolytes used in different sensors may be physically separated, for example to avoid dilution and/or contamination of the electrolytes of other sensors. In certain such embodiments, a barrier system can be used to provide independence of action, reaction, and/or signal. In some embodiments, the barrier system provides true physical and electrical isolation. In certain such embodiments, the truly isolated sensors cannot unintentionally connect to the electrodes of a different sensor or compromise the electrolyte of a different sensor, for example due to ion leakage across the barrier system.

In some embodiments, the barrier system may comprise an adhesive or glue system, for example comprising UV cure acrylics or RTV silicones as the barrier base material and a sealant applied to the catheter walls. Certain such embodiments may lack the bendability and flexibility generally desired to allow suitable introduction into veins and/or arteries. In some embodiments, the introduction of electrolytes may inhibit the adhesive from bonding to the housing, for example because the electrolyte pre-wets an interior wall of the housing, thereby allowing a potential ion path past the barrier system.

FIG. 3C illustrates an example embodiment of a portion 360 of a probe 40 comprising a barrier system. The barrier system comprises a first barrier 364a comprising a barrier material and a second barrier 364b comprising a barrier material. The barrier material may comprise, for example, a polymer such as butyl rubber, silicone rubber, or a soft durometer polymer, a monomer such as urethane, vinyl, rubber, or silicone gel, combinations thereof, and the like. The barriers 364a, 364b are in contact with an inner wall of a housing or cannula 361. The cannula 361 comprises a material that is permeable to the analyte to be measured in the portion 360. An electrolyte 363 is between the first barrier 364a and the second barrier 364b. A wire bundle or substrate (e.g., a flex circuit substrate) 362 extends through the barriers 364a, 364b. The portion 360 is physically and electrically isolated from portions proximate to and distal to the portion.

In some embodiments, a method of manufacturing the portion 360 comprises placing the substrate or wire bundle 362 into a molding apparatus. A barrier material is injected into the molding apparatus to form the first barrier 364a and the second barrier 364b. When the barriers 364a, 364b are inserted into the cannula 361, the barrier material forms mechanical contacts between the barriers 364a, 364b and the inner wall of the cannula 361. In certain embodiments, injecting the barrier material into the molding apparatus comprises forming one or more features 365 on the outer surface or diameter of the first barrier 364a and/or on the outer surface of the second barrier 364b. The features 365 may form a wiper action that can suitably seal and isolate electrolytes in adjacent portions from one another. The features 365 may also form air chambers that can isolate and/or interrupt ion exchange between portions since ions generally cannot traverse air.

In some embodiments, the cannula 361 may comprise an aperture (e.g., hole, slit, etc.) 366 in the outer wall of cannula 361, which can be used as a fill port for adding electrolyte 363. The aperture 366 can be formed during formation the cannula 361 or can be formed after formation of the cannula 361. The barrier system assembly is inserted into the cannula 361 just short of its final position so as to leave the aperture 366 in fluid communication with the space between the barriers 364a, 364b, allowing the electrolyte 363 to be injected into the portion 360 to fill the portion 360 with the electrolyte 363. The fluid previously in the portion 360 (e.g., comprising air) may be evacuated using a vacuum pump. Upon at least partial or complete filling of the portion 360 with the electrolyte 363, the barrier system assembly is slid into final position in which a barrier 364a, 364b at least partially covers or blocks the aperture 366.

FIG. 3D illustrates an example embodiment of a barrier system in which the barrier 364 comprises a chamber. A tube 368 connects the chamber 369 to the end of the probe 40. During insertion of the sensor assembly into the probe 40, the chamber 369 may be vacant (e.g., having been evacuated) to collapse the outer surface of the barrier 364 and to allow the sensor assembly to be easily inserted into the cannula 361. When the sensor assembly is in a desired position, the chamber 369 may be filled with a fluid (e.g., comprising air) to cause the barrier 364 to expand into place. In some embodiments, the chamber 369 is filled with the fluid until being at atmospheric or ambient pressure. In some embodiments, a fill tube 370 can be used to fill the sensor chamber with the electrolyte 363 after the barrier 364 is in position. In certain such embodiments, the sensor chamber may be filled with the electrolyte 363 after being at least partially evacuated.

FIG. 3E illustrates an example embodiment of a barrier system in which the cannula 361 is sealed from outside a rigid barrier 364 that is preassembled to the wire bundle or substrate 362. Each sensor chamber is filled with an electrolyte 363 by drawing the electrolyte 363 into the cannula 361 in a manner similar to filling a syringe. As the next barrier 364 enters cannula 361, the next sensor chamber is filled with an electrolyte 363, again in a manner similar to filling a syringe, until all chambers have been filled and the barriers 364 are in their final position within the cannula 361. After the sensor assembly is inserted into the cannula 361, the rigid barriers 364 are fused to cannula 361 by an external force as indicated by the arrow 371, for example comprising laser heating, ultrasonic heating, plasma heating, hot coil heating, combinations thereof, and the like.

FIG. 3F illustrates an example embodiment of a barrier system in which the barrier 364 comprises a central cavity 372 (e.g., comprising an arcuate chamber). In some embodiments, the cavity 372 is due to a feature 366 (FIG. 3C) extending around the circumference of a middle part of the barrier 364. In some embodiments, the cavity 372 is filled with an electrically insulating fluid 373 configured to provide sensor isolation. In some embodiments, the fluid 373 comprises oil, which may advantageously not produce condensed ambient water vapor in the form of dew that could provide a pathway for ion leakage and reduce electrical isolation between sensors.

FIG. 3G illustrates an example embodiment of a barrier system in which the barrier 364 is slightly smaller than the inner surface of the cannula 361 and in which the barrier 364 comprising a longitudinal gap. The sensor assembly may be easily inserted into the cannula 361 without resistance. A mold material 374 such as a polymer or monomer of suitable compliance, or an oil or gel, is then injected into the gap, sealing all surfaces simultaneously. In some embodiments, the mold material and/or the electrolyte can be injected via apertures in the cannula 361. In some embodiments, the mold material and/or the electrolyte can be injected using tubes inserted down the length of the cannula 361. In some embodiments, the mold material and/or the electrolyte can be applied as the sensors are inserted into the cannula 361. Other application methods are also possible.

In some embodiments, forming the cannula 361 comprises injection molding (e.g., gas-assisted injection molding). The injection molding may comprise forming pockets and applying a soft durometer monomer or polymer to the inside of the cannula 361 to seal the molded pockets. In certain embodiments, molding comprises forming two halves to form pockets, placing the sensor elements into the pockets, and then sealing the two halves (e.g., by heat sealing, glue sealing, ultrasonic sealing, combinations thereof, and the like).

Combinations of the barrier systems described herein and/or other barrier systems are also possible. In certain embodiments, the sealing systems herein may be advantageously used to form a probe 40 in which sensors are isolated and do not experience cross talk and/or leakage.

### Temperature Sensor

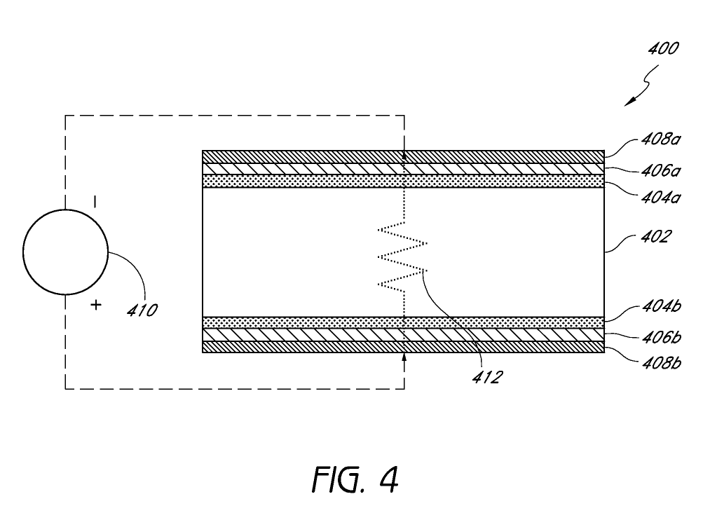

FIG. 4 illustrates an example of a temperature resistance detector (TRD) or temperature sensor 400. The sensor 400 comprises a substrate 402 comprising, for example, doped silicon (Si). Connection layers 404a, 404b comprising, for example, titanium (Ti) and/or tungsten (W), form a low ohmic contact on opposite sides of the substrate 402. The connection layers 404a, 404b may comprise the same material or different materials. Intermediate layers 406a, 406b comprising, for example, titanium, tungsten, and/or nickel (Ni), provide adhesion to interface layers 408a, 408b. The intermediate layers 406a, 406b may also inhibit diffusion or migration of material of the interface layers 408a, 408b into the connection layers 404a, 404b substrate 402. The intermediate layers 406a, 406b may comprise the same material or different materials. The interface layers 408a, 408b may comprise, for example, gold, aluminum, and/or silver. The interface layers 408a, 408b may comprise the same material or different materials.

The temperature of the sensor 400 may be determined by measuring the resistance of the sensor 400. In the illustrated embodiment, voltage from a voltage source 410 is applied between the interface layers 408a, 408b, and a current, traveling from the positive terminal of the voltage source 410 to the negative terminal of the voltage source 410, propagates through the interface layer 408b, then the intermediate layer 406b, then the connection layer 404b, then the substrate 402, then the connection layer 404a, then the intermediate layer 406a, and then the interface layer 408a, as illustrated by the dotted line 412. The voltage of the voltage source 410 is a known value and the current is measured, so the resistance R of the sensor 400 may be determined by application of Ohm's law, R=V/I, where V is voltage and I is current.

In certain materials, temperature is a function of resistance. The temperature of such materials may be calculated based on the measured resistance of the material. In some materials, temperature is a linear function of resistance over a certain temperature range. In certain such embodiments, the temperature T of a material can be calculated using the equation T=mR+b, where m is a slope constant, R is resistance, and b is an intercept constant. The slope constant m, or temperature coefficient of resistance (TCR), is at least partially based on the material of the substrate 402. For example, although platinum is not generally used in TRDs, the resistance change per unit of temperature of platinum is about 3,000 parts per million (ppm) over a broad temperature range. The layers 404a, 404b, 406a, 406b, 408a, 408b are more conductive than the substrate 402, so the resistance of the sensor 400 substantially and/or significantly depends on the resistance of the substrate 402. Temperature T may be calculated directly from the measured current I by combination with Ohm's law to produce the equation T=mV/I+b, where m, V, and b are known and/or constant.

The resistance of the substrate 402 is at least partially a function of thickness, surface area, and, for semiconductor substrates 402, dopant concentration. One or more of these parameters may be difficult to control in the substrate 402 of the sensor 400. For example, variations in dopant concentration from substrate 402 to substrate 402 or within (e.g., across, through) a single substrate 402 can cause resistance non-uniformities and/or gradients that can distort the measured resistance. Once a sensor 400 is fabricated, it may be difficult or impossible to adjust or calibrate the sensor 400 to determine values for the constants m and b described above. In some embodiments, it may be impractical to adjust or trim the sensor 400 to desired values of the constants m and/or b, for example by removing material, due to the different materials being used and/or the layering of the different materials. For example, removing material from the substrate 402 may cause a rough surface that renders proper ohmic contact with the one or both of the connection layers 404a, 404b difficult. A second resistor may be added to adjust the calibration, but the second resistor may affect the linearity of the relationship between temperature and resistance.

In some embodiments, the temperature sensor 400 is calibrated at room temperature (e.g., at about 25° C.). In some embodiments, the temperature sensor 400 is calibrated at about body temperature (e.g., at about 37° C.). Other calibration temperatures are also possible.

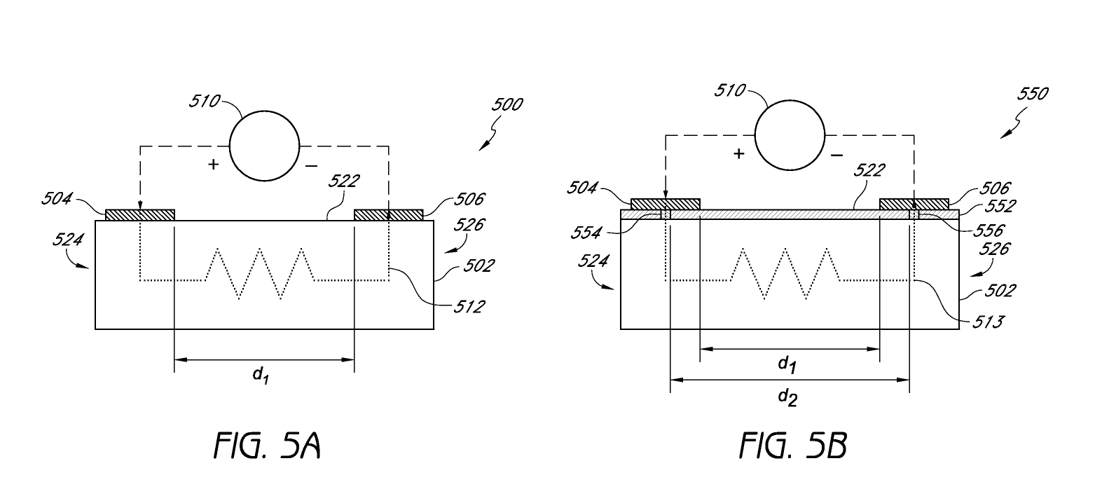

FIG. 5A illustrates an example embodiment of a temperature sensor 500. The sensor 500 comprises a substrate 502, a first contact 504, and a second contact 506. The substrate 502 comprises a first surface 522 having a lateral dimension. The first contact 504 is over the first surface 522 and is proximate to a first side 524 of the substrate 502. The second contact 506 is over the first surface 522 and is proximate to a second side 526 of the substrate 502. The second side 526 is opposite the first side 524 (e.g., being on an opposite side of the substrate 502). The second contact 506 is spaced from the first contact 504 by a first distance d₁.

In contrast to semiconductor devices comprising circuitry configured for logic, memory, etc., in which the substrate 502 is non-uniformly doped (e.g., to create p-n junctions, wells, etc.), the substrate 502 is substantially uniformly doped. In some embodiments, the substrate 502 comprises doped silicon (e.g., n-doped with elements such as phosphorous (P), arsenic (As), and/or antimony (Sb); p-doped with elements such as boron (B) and/or aluminum). In some embodiments, the substrate 502 comprises doped semiconductor material such as gallium arsenide (GaAs), germanium (Ge), carbon (C), combinations thereof, and the like. In some embodiments, after doping, the resistance of the substrate 502 is at least about 125 ohms per cubic centimeter (Ω/cm³).

The first contact 504 comprises a material that is different from the material of the substrate 502. In some embodiments, the material of the first contact 504 is more conductive than the material of the substrate 502. In certain such embodiments, the first contact 504 comprises aluminum, copper, nickel, platinum, gold, silver, alloys thereof, combinations thereof, and the like. The second contact 506 comprises a material that is different from the material of the substrate 502. In some embodiments, the material of the second contact 506 is more conductive than the material of the substrate 502. In certain such embodiments, the second contact 506 comprises aluminum, copper, nickel, platinum, gold, silver, alloys thereof, combinations thereof, and the like. The second contact 506 may comprise the same material as the first contact 504 or a material that is different from the material of the first contact 504. As described in further detail below, the first contact 504 and the second contact 506 may be formed over the surface 526 of the substrate 502 without a connecting layer such as epoxy.

In the illustrated embodiment, voltage from a voltage source 510 is applied between the first contact 504 and the second contact 506, and a current, traveling from the positive terminal of the voltage source 510 to the negative terminal of the voltage source 510, propagates through the first contact 504, then the substrate 502, and then the second contact 506, as illustrated by the dotted line 512. The first contact 504 and the second contact 506 are more conductive than the substrate 502, so the resistance of the sensor 500 substantially and/or significantly depends on the resistance of the substrate 502. The resistance of the substrate 502 of the sensor 500 is at least partially a function of the distance d₁ between the first contact 504 and the second contact 506. In some embodiments, the current propagates through a substantial portion of the substrate 502. For example, in contrast to semiconductor devices comprising circuitry, in which current only propagates through a small portion of the substrate 502 (e.g., a gate), current passes though the bulk of the substrate 502. In certain such embodiments, the distance d₁ may be greater than about 75% of the lateral dimension of the first surface 522 of the substrate 502, greater than about 85% of the lateral dimension of the first surface 522 of the substrate 502, greater than about 90% of the lateral dimension of the first surface 522 of the substrate 502, or greater than about 95% of the lateral dimension of the first surface 522 of the substrate 502.

The resistance of the substrate 502 is at least partially based on the distance d₁ between the first contact 504 and the second contact 506 and the thickness of the substrate 502. In some embodiments in which a probe 40 comprising the sensor 500 is configured to be implanted or inserted into a blood vessel, thickness of the sensor 500, and thus the substrate 502, is limited (e.g., not easily adjusted). For example, a thickness of the substrate 502 may be about 100 micrometers or microns (μm). In certain embodiments, the resistance of the sensor 500 can be increased by increasing the distance d₁ between the first contact 504 and the second contact 506. Increasing resistance of the substrate 502 may increase the sensitivity and accuracy of the sensor 500 to temperature changes. By contrast, increasing the lateral dimension of the substrate 402 of the sensor 400 would decrease resistance and may decrease the sensitivity and accuracy of the sensor 400 to temperature changes. In some embodiments, the resistance of the sensor 500 may be at least about ten times greater than the resistance of the sensor 400. In some embodiments, the TCR of the substrate 502 is at least about 4,250 ppm. In certain embodiments, the resistance of the substrate 502 to the current is substantially linearly proportional to the temperature of the substrate 502 between about 33° C. and about 41° C. In some embodiments, resistance of the sensor 500 can be calculated within one-hundredth to one-thousandth of an ohm.

FIG. 5B illustrates another example embodiment of a temperature sensor 550. The temperature sensor 550 comprises a substrate 502, a first contact 504, and a second contact 506. In some embodiments, the substrate 502, the first contact 504, and the second contact 506 are similar to those described above with respect to the temperature sensor 500 of FIG. 5A. The temperature sensor 550 further comprises an insulating layer 552, a first via 554, and a second via 556.

The insulating layer 552 is between the first surface 522 of the substrate 502 and the first contact 504 and is between the first surface 522 of the substrate 502 and the second contact 506. Although the insulating layer 552 is illustrated as being above the first surface 522 of the substrate 502 between the first contact 504 and the second contact 506, the insulating layer only be between the first surface 522 of the substrate 502 and the first contact 504 and between the first surface 522 of the substrate 502 and the second contact 506. The insulating layer 552 comprises an electrically insulating material such as, for example, silicon oxide (SiOₓ (e.g., SiO₂)), silicon nitride (SiNₓ (e.g., Si₃N₄)), silicon oxynitride (SiOₓN_{y} (e.g., SiON)), aluminum oxide (AlOₓ (e.g., Al₂O₃)), combinations thereof, and the like.

The first via 554 is through the insulating layer and electrically connects the first contact 504 and the substrate 502. The first via 554 comprises a conductive material such as aluminum, copper, nickel, platinum, gold, silver, tin-silver solder, tin-silver-copper solder, alloys thereof, combinations thereof, and the like. In some embodiments, the first via 554 comprises the same material as the first contact 504. The second via 556 is through the insulating layer and electrically connects the second contact 506 and the substrate 502. The second via 556 comprises a conductive material such as aluminum, copper, nickel, platinum, gold, silver, tin-silver solder, tin-silver-copper solder, alloys thereof, combinations thereof, and the like. In some embodiments, the second via 556 comprises the same material as the second contact 506.

In the illustrated embodiment, voltage from a voltage source 510 is applied between the first contact 504 and the second contact 506, and a current, traveling from the positive terminal of the voltage source 510 to the negative terminal of the voltage source 510, propagates through the first contact 504, then the first via 554, then the substrate 502, then the second via 556, and then the second contact 506, as illustrated by the dotted line 513.

The second via 556 is spaced from the first via 552 by a distance d₂. The resistance of the substrate 502 of the sensor 500 is at least partially a function of the distance d₂ between the first via 554 and the second via 556. In some embodiments, the current propagates through a substantial portion of the substrate 502. For example, in contrast to semiconductor devices comprising circuitry, in which current only propagates through a small portion of the substrate 502 (e.g., a gate), current passes though the bulk of the substrate 502. In certain such embodiments, the distance d₂ may be greater than about 75% of the lateral dimension of the first surface 522 of the substrate 502, greater than about 85% of the lateral dimension of the first surface 522 of the substrate 502, greater than about 90% of the lateral dimension of the first surface 522 of the substrate 502, or greater than about 95% of the lateral dimension of the first surface 522 of the substrate 502.

The resistance of the substrate 502 of the sensor 550 is at least partially based on the distance d₂ between the first via 554 and the second via 556 and the thickness of the substrate 502. In some embodiments in which a probe 40 comprising the sensor 550 is configured to be implanted or inserted into a blood vessel, thickness of the sensor 550, and thus the substrate 502, is limited (e.g., not easily adjusted). For example, a thickness of the substrate 502 may be about 100 micrometers or microns (μm). In certain embodiments, the resistance of the sensor 550 can be increased by increasing the distance d₂ between the first via 554 and the second via 556. Increasing resistance of the substrate 502 may increase the sensitivity and accuracy of the sensor 550 to temperature changes. In some embodiments, the resistance of the sensor 550 may be at least about ten times greater than the resistance of the sensor 400. In some embodiments, the TCR of the substrate 502 is at least about 4,250 ppm per unit temperature. In certain embodiments, the resistance of the substrate 502 to the current is substantially linearly proportional to the temperature of the substrate 502 between about 33° C. and about 41° C. In some embodiments, resistance of the sensor 550 can be calculated within one-hundredth to one-thousandth of an ohm.

Material from the first contact 504 and the second contact 506 may diffuse or migrate into the substrate 502, for example due to concentration gradients, entropy, Fick's laws, etc. Diffusion can reduce the accuracy of the sensor 500, 550 by changing the linearity and resistivity of a portion of the substrate 502. Diffusion is at least partially a function of contact area, temperature, and time. In the sensor 550, the first via 554 and the second via 556 reduce the contact area between the material of the first contact 504 and the second contact 506, respectively, and the substrate 502, thereby reducing diffusion. If material from the first contact 504 and/or the second contact 506 diffuses into the insulating layer 552, the resistivity of the substrate 502, and thus the accuracy of the sensor 550, is not affected.

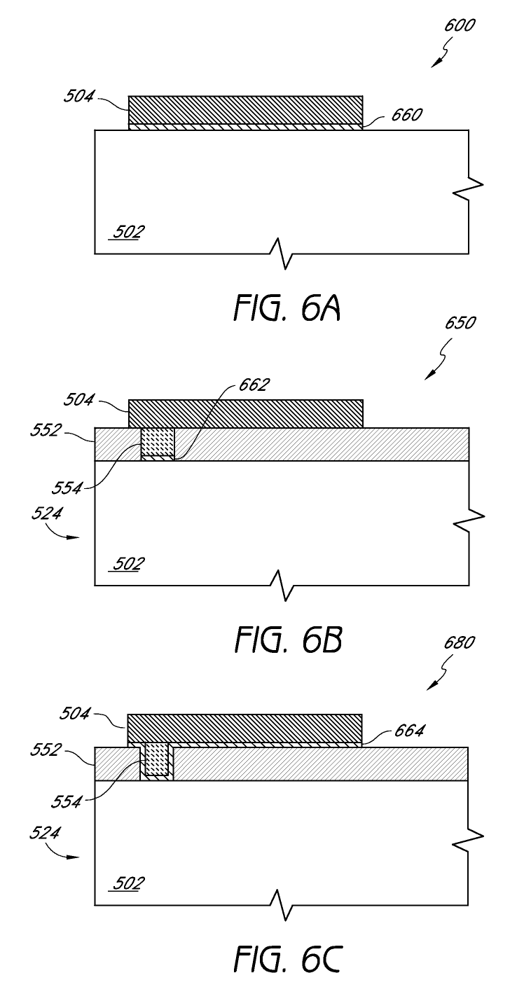

FIGS. 6A, 6B, and 6C are cutaway and cross-sectional views of a portion of example embodiments of a temperature sensor 600, a temperature sensor 650, and a temperature sensor 680, respectively. The temperature sensor 600 comprises a substrate 502, a first contact 504, and a second contact (not shown). In some embodiments, the substrate 502, the first contact 504, and the second contact are similar to those described above with respect to the temperature sensor 500 of FIG. 5A.

The temperature sensor 600 further comprises a barrier metal layer 660 between the first contact 504 and the substrate 502 and between the second contact and the substrate 502 (not shown). Although the barrier metal layer 660 is described herein as being between the first contact 504 and the substrate 502 and between the second contact and the substrate 502, the barrier metal layer 660 may also be characterized as being a portion of the first contact 504 and the second contact. The barrier metal layer 660 comprises molybdenum (Mo), tungsten, titanium, tantalum (Ta), nitrides thereof, alloys thereof, combinations thereof, and the like. In some embodiments, the barrier metal layer 660 has a thickness that is thick enough that it blocks the diffusion path between the material from the first contact 504 into the substrate 502 and from the second contact into the substrate 502, but that is thin enough that it does not substantially and/or significantly increase resistance between the first contact 504 and the substrate 502 and between the second contact and the substrate 502. In some embodiments, the barrier metal layer 660 increases adhesion between the first contact 504 and the substrate 502 and between the second contact and the substrate 502.

The temperature sensor 650 and the temperature sensor 680 each comprise a substrate 502, a first contact 504, a second contact (not shown), an insulating layer 552, a first via 554, and a second via (not shown). In some embodiments, the substrate 502, the first contact 504, the second contact, the insulating layer 552, the first via 554, and the second via are similar to those described above with respect to the temperature sensor 500 of FIG. 5A and/or the temperature sensor 550 of FIG. 5B.

The temperature sensor 650 further comprises a barrier metal layer 662 between the first via 554 and the substrate 502 and between the second via and the substrate 502 (not shown). In comparison to the temperature sensor 550 of FIG. 5B, the temperature sensor 650 also reduces diffusion of the material of the first contact 504 into the substrate 502 and of the second contact 506 into the substrate 502 because the contact area therebetween is reduced by the first via 554 and the second via 556, and the barrier metal layer 662 further reduces diffusion of the material of the first contact 504 into the substrate 502 and of the second contact 506 into the substrate 502 by blocking the diffusion path. In comparison to the temperature sensor 600 of FIG. 6A, the temperature sensor 650 also comprises a barrier metal layer 662 that reduces diffusion of the material of the first contact 504 into the substrate 502 and of the second contact 506 into the substrate 502 by blocking the diffusion path, and the first via 554 and the second via can provide a more consistent path through the substrate 502 and/or allows increasing the resistance of the substrate 502 by increasing the distance d₂ between the first via 554 and the second via.

The temperature sensor 680 further comprises a barrier metal layer 664 between the first via 554 and the substrate 502 and between the second via and the substrate 502 (not shown). The barrier metal layer 664 is also between the first contact 506 and the insulating layer 552 and is between the second contact and the insulating layer 552. In comparison to the temperature sensor 650 of FIG. 6B, the temperature sensor 680 also reduces diffusion of the material of the first contact 504 into the substrate 502 and of the second contact 506 into the substrate 502 because the contact area therebetween is reduced by the first via 554 and the second via 556, further reduces diffusion of the material of the first contact 504 into the substrate 502 and of the second contact 506 into the substrate 502 by blocking the diffusion path, the first via 554 and the second via can provide a more consistent path through the substrate 502 and/or allows increasing the resistance of the substrate 502 by increasing the distance d₂ between the first via 554 and the second via, and the barrier metal layer 664 further reduces diffusion of the material of the first contact 504 into the insulating layer 552 and of the material of the second contact into the insulating layer such that the material of the first contact 504 and the second contact 506 is inhibited from eventually diffusing into the substrate 502. In some embodiments, the barrier metal layer 664 may also reduce manufacturing complexity, as described in further detail below.

In some embodiments, for example as illustrated in FIG. 5B, the first via 554 is near a center or middle of the first contact 504, for example to reduce manufacturing complexity by increasing overlay margins. In some embodiments, for example as illustrated in FIGS. 6A through 6C, the first via 554 is proximate to the first side 524 of the substrate 502, for example to increase the distance d₂ and thus the resistance of the substrate 502 and the accuracy of the sensor 550. It will be appreciated that the second via 556 may be near a center or middle of the second contact 506 or proximate to the second side 526 of the substrate 526, and that the position of the via is independent of the existence or non-existence of the barrier layer 660.

In certain embodiments, a method of manufacturing a temperature sensor begins with a p-type substrate (e.g., a silicon wafer doped with boron). In certain embodiments, a method of manufacturing a temperature sensor begins with an n-type substrate (e.g., a silicon wafer doped with phosphorous). In certain embodiments, a method of manufacturing a temperature sensor begins with an undoped substrate (e.g., an undoped silicon wafer). The substrate is then substantially uniformly doped. In some embodiments, doping the substrate comprises thermal doping, electron beam scanning, or neutron bombardment. In certain embodiments in which the starting substrate is n-type, doping can change the substrate to be substantially uniformly p-type or more n-type (e.g., n⁺, n⁺⁺). In certain embodiments in which the starting substrate is p-type, doping can change the substrate to be substantially uniformly n-type or more p-type (e.g., p⁺, p⁺⁺). In certain embodiments in which the starting substrate is undoped, doping can change the substrate to be substantially uniformly n-type or p-type. In some embodiments, driving the dopant into the substrate by heating may induce variations in dopant concentration. In some embodiments, after doping, the resistance of the substrate is at least about 125 ohms per cubic centimeter (Ω/cm³). In certain embodiments, neutron bombarded material, which can be obtained from, for example, GE Sensors, may have a more uniform dopant concentration, which can improve the bulk resistance uniformity as well as the resistance value from one substrate to the next.

In embodiments in which the sensor comprises an insulating layer (e.g., the temperature sensors 550, 650, 680 described herein), the substantially uniformly doped substrate may be placed into a diffusion furnace to grow a thermal oxide layer that coats the surface with an insulation layer 552. Deposition of an oxide layer and other insulating materials are also possible. The vias through the insulating layer 552 may be formed using photolithography and wet and/or dry etching that remove portions of the insulating layer to allow contact between the substantially uniformly doped substrate 502 and the contacts 504, 506. Other patterning techniques are also possible.

In embodiments in which the sensor comprises a barrier layer (e.g., the temperature sensors 600, 650, 680, the barrier layer 600, 662, 664 may be deposited. In some embodiments, for example in which the temperature sensor does not comprise an insulating layer 552, a barrier layer 600 may be blanket deposited and then patterned with the contacts 504, 506. In some embodiments, for example in which the temperature sensor comprises an insulating layer 552, a barrier layer 662 may be selectively deposited on exposed areas of the substrate 502. In some embodiments, for example in which the temperature sensor comprises an insulating layer 552, a barrier layer 664 may be blanket deposited and then patterned with the contacts 540, 506. A barrier layer may provide a low resistance interface between the contacts 504, 506 and the substrate 502. A resistance at the contact interface that is different than the resistance of the substrate may result in non-linear performance. A barrier layer may also increase adhesion between the contacts 504, 506 and the substrate 502 and/or between the contacts 504, 506 and the insulating layer 552.

The contacts 504, 506 may be formed by coating the substrate 502 with a contact layer material (e.g., comprising one or more metal layers) and then using traditional photolithography and wet and/or dry etching to remove contact layer material from non-contact areas. Other patterning techniques are also possible. In some embodiments, the device is stabilization baked to cause diffusion, which can limit drift during use (e.g., because the sensor can be calibrated with the diffusion having already taken place).

In some embodiments, a temperature sensor 500, 550, 600, 650, 680 may be manufactured as described herein and then mounted on a flex circuit. In some embodiments, the manufacturing processes described herein for forming a resistive temperature sensor may be at least partially performed directly on a flex circuit.

The layout of the temperature sensors 500, 550, 600, 650, 680 in which the contacts 504, 506 are on the same surface of the substrate 502 and have a distance d₁, d₂ therebetween that increases the circuit resistance can reduce (e.g., greatly reduce) power consumption and/or can produce good sensitivity to temperature changes.

Although structurally different from the temperature sensor 400, the temperature sensor 500, the temperature sensor 550, the temperature sensor 600, the temperature sensor 650, and the temperature sensor 680 may be calibrated and/or used to determining temperature based on a measured current propagating therethrough, which is indicative of resistivity, as described above with respect to the temperature sensor 400 (e.g., using a linear calculation).

Although not depicted in the Figures, the temperature sensors 500, 550, 600, 650, 680 may be at least partially immersed in or surrounded by a fluid (e.g., a fluid having a low heat capacity or a fluid having a heat capacity similar to blood). In some embodiments, the size of the sensor portion comprising the temperature sensor 500, 550, 600, 650, 680 may be reduced or minimized in order to reduce or minimize the volume of the fluid so that the amount of heat energy transferred to or from the blood to reflect a change in blood temperature is reduced or minimized. In some embodiments, the temperature sensors 500, 550, 600, 650, 680 may be surrounded by air or another gas.

### Gas Concentration Sensors

Sensors for measuring the concentrations of various gases dissolved in fluids such as blood may be of two types: actively driven (polarographic) or galvanometric. To operate actively driven sensors, electronic circuitry maintains a desired potential difference between a pair of electrodes, a cathode and an anode comprising the same or similar conductive material, suspending in and exposed to an electrolyte, while measuring the flow of current between two electrodes that may be the same or different than the electrodes to which the potential is applied. The magnitude of the measured current is proportional to the concentration of gas in the electrolyte, which, in turn, depends on the partial pressure of the gas in the fluid surrounding the sensor. To operate galvanometric sensors, electronic circuitry monitors the potential difference between a pair of electrodes, a cathode and an anode of comprising different or dissimilar conductive materials, suspended in and exposed to an electrolyte. The magnitude of the measured potential or voltage is proportional to the concentration of gas in the electrolyte, which, in turn, depends on the partial pressure of the gas in the fluid surrounding the sensor.

For oxygen concentration measurement in a galvanometric sensor comprising fluid saline (NaCl) electrolyte where the cathode comprises gold and the anode comprises silver, the electrochemical reduction reaction at the working electrode or cathode can be described as:

O_{2(g)}+2H₂O₍ₗ₎+4e⁻→4OH⁻_{(aq)}

The electrochemical oxidation reaction at the counter electrode or anode can be described as:

Ag₍ₛ₎+Cl⁻_{(aq)}→AgCl₍ₛ₎+e⁻

For oxygen concentration measurement in an actively driven sensor comprising fluid sodium hydroxide (NaOH) electrolyte where the cathode and the anode both comprise platinum, the electrochemical reduction reaction at the working electrode or cathode can be described as:

O_{2(g)}+2H₂O^{(l)}+4e⁻→4OH⁻_{(aq)}

The electrochemical oxidation reaction at the counter electrode or anode can be described as:

4OH⁻_{(aq)}→O_{2(g)}+2H₂O₍ₗ₎+4e⁻

In embodiments in which the materials for the cathode, anode, and/or electrolyte are different from those discussed above, the reduction and oxidation reactions will be different in detail, but, for all, electrons combine with oxygen gas to form anions at the cathode and anions with or without an oxygen atom undergo a reaction to release electrons to the anode. The number of electrons involved in the reactions is directly proportional to the concentration of oxygen in the electrolyte, which is evident in the voltage or current being generated by the sensor.

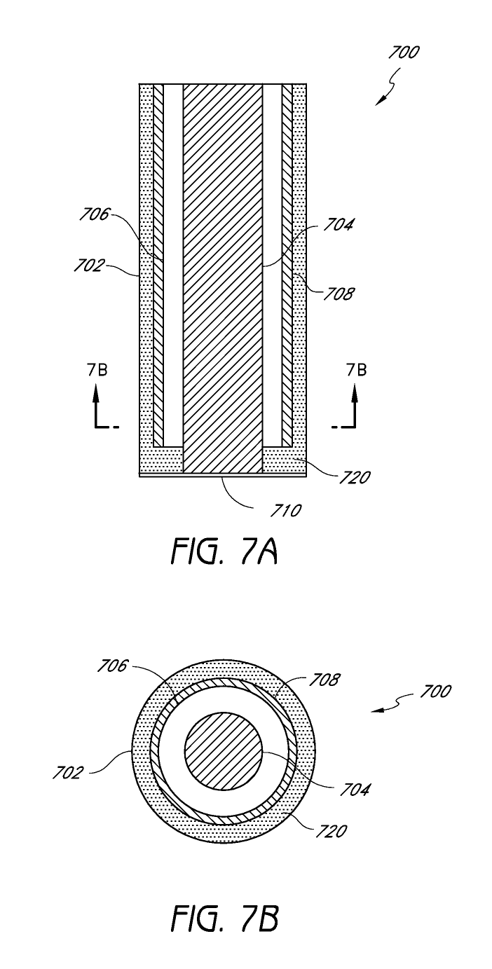

The major difference between the two types of sensors is that a galvanic oxygen sensor generates a voltage whereas a polarographic oxygen sensor, if supplied with a small voltage of the order of about 0.8 volts, generates a current. Each of these sensors has strengths and weaknesses; at least one aspect of the inventions described herein is the recognition that the strengths of each type may be used to offset the weaknesses of the other type. FIGS. 7-10 illustrate example geometries that can function effectively for either type of oxygen sensor. Each type of sensor uses a different combination of electrode materials and electrolytes in order to produce a signal that is proportional to the dissolved oxygen in the fluid surrounding the sensor, but all described geometries can be utilized for sensing oxygen.

In some embodiments, an implantable sensor subassembly for measuring blood oxygen concentration comprises at least one of a galvanometric sensor and a polarographic sensor, for example the sensors described herein. Other types of galvanometric sensors and polarographic sensors are also possible. Signal values from one or both of the galvanometric sensor and the polarographic sensor can be utilized to measure the partial pressure of oxygen in the blood.

In certain embodiments, a galvanometric sensor comprises a plurality of electrodes suspended and separated slightly from each other in an electrolyte configured to support an electrochemical reaction (e.g., the electrochemical reactions described above). The electrodes and the electrolyte are at least partially suspended in a cell that is permeable to oxygen (e.g., comprising a sealed segment of a plastic or polymer tube that is permeable to oxygen). The electrodes comprise electrochemically different materials such as, for example: gold and zinc; nickel and cadmium; copper and nickel; and the like. When suspended in the electrolyte, the electrodes generate a voltage that is monotonically dependent upon the oxygen concentration in the electrolyte. In some embodiments, the electrodes are suspended using a substrate or structure comprising silicon, plastic, polymer, ceramic, other suitable insulating material, or combinations of insulating and/or non-insulating materials.

In certain embodiments, a polarographic sensor comprises a plurality of electrodes suspended and separated slightly from each other in an electrolyte configured to support an electrochemical reaction (e.g., the electrochemical reactions described above). The electrodes and the electrolyte are at least partially contained in a cell that is permeable to oxygen (e.g., comprising a sealed segment of a plastic or polymer tube that is permeable to oxygen). The electrodes comprise materials that are substantially electrochemically identical such as, for example: gold and gold; platinum and platinum; and the like. The materials of the electrodes may be different if they are substantially electrochemically identical. When suspended in the electrolyte and provided with an appropriate voltage, the electrodes generate a current that is monotonically dependent upon the oxygen concentration in the electrolyte. In some embodiments, the electrodes are suspended using a substrate or structure comprising silicon, plastic, polymer, ceramic, other suitable insulating material, or combinations of insulating and/or non-insulating materials.

The term “drift” may be used to describe any change in oxygen sensor output (voltage or current) as a function of time that is not caused by a concomitant change in the oxygen concentration in the fluid external to the sensor. Galvanic oxygen sensors can generally respond very quickly and drift slowly as the sensor is operated and as the electrode and electrolyte materials are affected by the current flowing through the sensor. Polarographic oxygen sensors can generally respond more slowly and drift even more slowly as the sensor is operated and as the electrode and electrolyte materials are affected by the current flowing through the sensor. Electronics and/or algorithms used for signal analysis computation may compensate for drift parameters by using predictive and/or combinatoric methods based on experimental results, such that the resulting oxygen concentration measured by the oxygen sensor or sensor combination is accurate for the intended operational duration of the probe 40. To the extent that said values drift over a period of time independent of any change in oxygen concentration, this drift may be compensated by predictive voltage and/or current analysis algorithms embedded in the electronics (e.g., in the display module) to yield an accurate measurement of blood oxygen concentration during the period of operation of the sensors.

In some embodiments, a measured temperature (e.g., from the temperature sensors described herein) can be used to adjust the calculation of gas concentration and/or pH. For example, formulae, algorithms, tables, combinations thereof, and the like may be used to compensate for the increased or reduced activity of a gas sensor at lower or higher blood temperatures.

U.S. patent application Ser. Nos. 10/658,926 and 12/172,181 and U.S. Provisional Patent App. No. 61/196,706, each of which is hereby incorporated by reference in its entirety, disclose further details example embodiments of gas concentration sensors. Embodiments in which a probe comprises combinations of sensors described herein and/or incorporated by reference are also possible.

FIG. 7A illustrates an example of a concentration sensor 700. FIG. 7B is a cross-sectional view of the sensor 700 of FIG. 7A taken along the line 7B-7B. The sensor 700 comprises an electrically insulating housing 702 (e.g., comprising plastic or polymer) at least partially containing a first or sensing or working electrode 704, a second or counter or reference electrode 708, an insulator 706 between the sensing electrode 704 and the reference electrode 708, and an electrolyte solution 720. The electrolyte 720 may comprise saline fluid or another fluid, gel, or solid that is permeable to oxygen and that can support the desired electrochemical reactions. The sensing electrode 704 comprises a first material and the reference electrode 708 comprises a second material different than the first material. The electrodes 704, 708 are both in contact with the electrolyte 720. The dissimilarity of the materials of the electrodes 704, 708 creates a voltage proportional to the concentration of certain gases (e.g., oxygen) in the electrolyte 720, which depends on the concentration of those gases in the fluid surrounding the sensor 700.

As best illustrated in FIG. 7B, the sensing electrode 704 and the reference electrode 708 may be coaxial, and the housing 702 may be impermeable to gas. The sensing electrode 704 is substantially cylindrical. The insulator 706 surrounds the sensing electrode 704. The reference electrode 708 surrounds the insulator 706. Referring again to FIG. 7A, the sensing electrode 704 extends beyond the insulator 706 and the reference electrode 708 and may be directly in contact with a membrane 710. The membrane 710 is permeable to the gas to be measured (e.g., oxygen) and is in contact on one side with blood or with a structure that is in direct contact with the blood and that is also permeable to the gas to be measured, such that the desired reactions, depending on concentration of the gas, can proceed. The gas molecules permeate through the membrane 710 into the electrolyte 720, where they can interact with the electrodes 708, 708 to cause the desired electrochemical reactions, which are dependent on gas concentration.

Certain galvanic sensors can immediately reach equilibrium and have virtually no warm-up time. Galvanic sensors may achieve good sensitivity and accurate readings, for example because there is no applied potential difference that may drift and cause the measured current to drift.

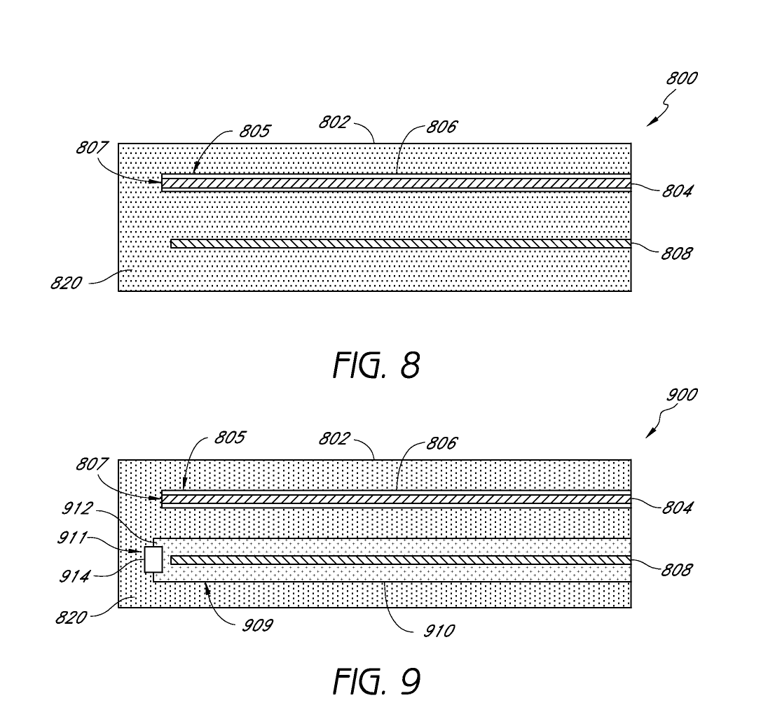

FIG. 8 is a cross-sectional view of another example embodiment of a blood gas concentration sensor 800. The sensor 800 comprises a first housing 802 at least partially defining a first chamber containing a first electrolyte 820. The first housing 802 comprises a gas permeable material. The sensor 800 further comprises a first or sensing electrode 804 and a second or reference electrode 808. The first electrode 804 comprises sides 805 and an end 807. The sides 805 of the first electrode 804 are surrounded by a first insulating later 806. The insulating layer 806 is electrically insulating and gas impermeable. The end 807 of the first electrode 804 is in contact with the first electrolyte 820. The end 807 of the first electrode 804 is not in contact with the first housing. The first electrode 804 comprises a first metal. In certain embodiments, the first metal comprises nickel, cadmium, iron, chromium, zinc, manganese, aluminum, beryllium, or magnesium. In some embodiments, the first electrode 804 comprises an insulated wire having a cut end. In certain such embodiments, the cross-section of the first electrode 804 or other small portion of the first electrode 804 is in contact with the first electrolyte 820. The second electrode 808 is substantially parallel to the first electrode 804. The second electrode 808 comprises a second metal. In certain embodiments, the second metal comprises copper, silver, palladium, platinum, or gold. In some embodiments, the second electrode 808 is in contact with the first electrolyte 820 along all or a portion of its length within the sensor 800. In some embodiments, the second electrode 808 comprises an uninsulated wire or rod. The electrodes 804, 808 are configured to allow the desired electrochemical reactions to proceed. A potential difference between the first metal and the second metal is at least about 0.5 volts. In some embodiments, the electrodes 804, 808 comprise thin metal films placed or deposited on a thin insulating film or structure (e.g., comprising plastic, polymer, ceramic, or semiconductor).

FIG. 9 is a cross-sectional view of another example embodiment of a blood gas concentration sensor 900. The sensor 900 further comprises a second housing 910 at least partially in the first chamber. The second housing 910 comprises sides 909 and an end 911 comprising a first frit 914. The second housing 910 is electrically insulating and gas impermeable (e.g., comprising polyimide or glass). The sides 909 and the end 911 of the second housing 910 at least partially define a second chamber containing a second electrolyte 912. The second electrode 808 is in contact with the second electrolyte 912. In some embodiments, the second housing 910 comprises a cylinder. In some embodiments, the housing 910 comprises a wall. For example, in embodiments in which the electrodes 804, 808 are on a flat surface (e.g., comprising silicon), the second housing 910 may comprise a wall or channel of silicon. The first frit 914 may comprise a porous material (e.g., comprising a porous glass such as Vycor® 7930, available from Corning, Inc. of Corning, N.Y.). The first frit 914 may comprise a polymer, gel, or even silicon. The pores of first frit 914 may be filled with a combination of the first electrolyte 820 and the second electrolyte 912 so that an electrically active junction is formed for transport of cations and/or anions. The second electrolyte 912 may comprise a fluid, gel, or solid that supports the electrochemical reaction at the second electrode 808. If the first electrolyte 820 and the second electrolyte 912 both comprise liquids or semi-solid liquids, this may be termed a “liquid junction.”

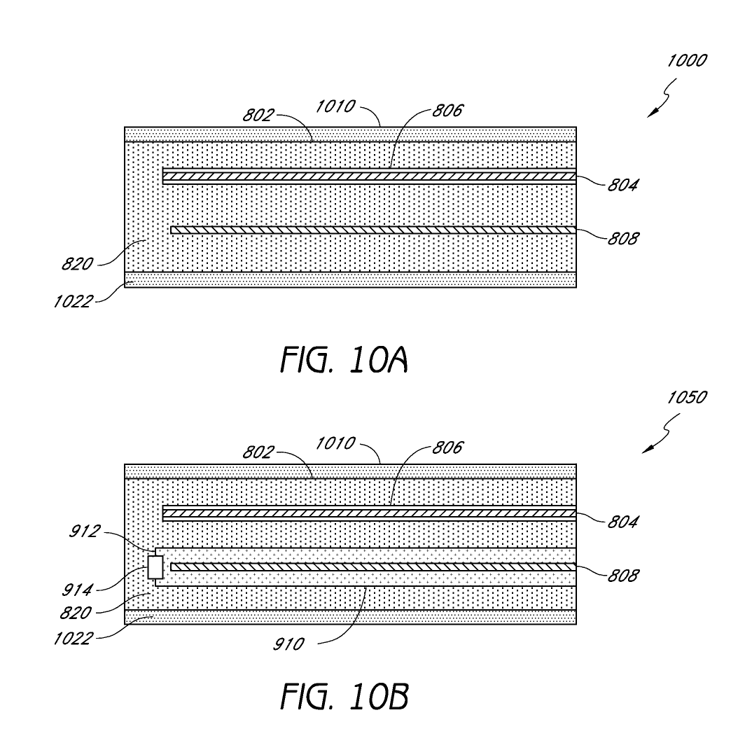

FIG. 10A is a cross-sectional view of yet another example embodiment of a blood gas concentration sensor 1000. In comparison to the sensor 800 depicted in FIG. 8, the sensor 1000 comprises a second housing 1010 at least partially defining a second chamber containing a second electrolyte 1022. The first housing 802 is at least partially in the second chamber. The second housing 1010 may comprise a non-porous membrane configured to provide appropriate support and permeability for the sensor 1000.

FIG. 10B is a cross-sectional view of still another example embodiment of a blood gas concentration sensor 1050. In comparison to the sensor 900 depicted in FIG. 9, the sensor 1050 comprises a third housing 1010 at least partially defining a third chamber containing a third electrolyte 1022. The first housing 802 is at least partially in the third chamber. The third housing 1010 may comprise a porous or non-porous membrane configured to provide appropriate support and permeability for the sensor 1050.

Although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Thus, it is intended that the scope of the invention herein disclosed should not be limited by any particular embodiment(s) described above.

What is claimed is:

1. An implantable galvanometric sensor for measuring blood gas concentration, the sensor comprising:
a first gas permeable tube at least partially defining a first chamber containing a first electrolyte;
a second gas permeable tube at least partially in the first chamber and at least partially defining a second chamber containing a second electrolyte;
a first sensing electrode extending into the second chamber from a first direction, the first sensing electrode comprising a first insulated wire having an exposed end in contact with the second electrolyte and not in contact with the second gas permeable tube, the exposed end of the first insulated wire being a substantially radial cross-section of the first insulated wire, the first sensing electrode comprising a first metal;
a third tube at least partially in the second chamber, the third tube comprising sides and an end comprising a first frit, the sides of the third tube being gas impermeable, the sides and the end of the third tube at least partially defining a third chamber containing a third electrolyte; and
a first reference electrode in the third chamber and extending into the second chamber from the first direction, the first reference electrode substantially parallel to the first sensing electrode, the first reference electrode comprising a second metal, wherein a potential difference between the first metal and the second metal is at least about 0.5 volts.

2. The sensor of claim 1, wherein the sensing electrode comprises gold and the reference electrode comprises zinc.

3. The sensor of claim 1, wherein the first gas permeable tube at least partially defines a fourth chamber containing a fourth electrolyte, the fourth chamber longitudinally spaced from the first chamber by an insulating adhesive, the sensor further comprising:
a second sensing electrode extending into the fourth chamber in a second direction, the second sensing electrode comprising a second insulated wire having an exposed end in contact with the fourth electrolyte and not in contact with the fourth gas permeable tube, the exposed end of the second insulated wire being a substantially radial cross-section of the second insulated wire, the second sensing electrode comprising a third metal;
a fourth tube at least partially in the fourth chamber, the fourth tube comprising sides and an end comprising a second frit, the sides of the fourth tube being gas impermeable, the sides and the end of the fourth tube at least partially defining a fifth chamber containing a fifth electrolyte;
a second reference electrode extending into the fifth chamber in the second direction and substantially parallel to the second sensing electrode, wherein a potential difference between the third metal and the fourth metal is at least about 2.5 volts.

4. The sensor of claim 3, wherein the second direction is the same as the first direction.

5. The sensor of claim 3, wherein the second sensing electrode comprises gold and the second reference electrode comprises magnesium.

6. A blood gas concentration sensor comprising:
a first housing at least partially defining a first chamber containing a first electrolyte, the first housing comprising a gas permeable material;
a first electrode in the first chamber, the first electrode comprising sides and an end, the sides of the first electrode surrounded by a first insulating layer, the end of the first electrode in contact with the first electrolyte, the end of the first electrode not in contact with the first housing, the first electrode comprising a first metal; and
a second electrode in the first chamber and substantially parallel to the first electrode, the second electrode comprising a second metal, wherein a potential difference between the first metal and the second metal is at least about 0.5 volts.

7. The sensor of claim 6, wherein the second electrode is in contact with the first electrolyte.

8. The sensor of claim 6, further comprising a second housing at least partially defining a second chamber containing a second electrolyte, the first housing at least partially in the second chamber.

9. The sensor of claim 6, further comprising a second housing at least partially in the first chamber, the second housing comprising sides and an end comprising a first frit, the second housing being gas impermeable, the sides and the end of the second housing at least partially defining a second chamber containing a second electrolyte, wherein the second electrode is in the second chamber and is in contact with the second electrolyte.

10. The sensor of claim 9, wherein the sides of the second housing comprise polyimide or glass.

11. The sensor of claim 9, further comprising a third housing at least partially defining a third chamber containing a third electrolyte, the first housing at least partially in the third chamber.

12. The sensor of claim 6, wherein the first metal comprises nickel, cadmium, iron, chromium, zinc, manganese, aluminum, beryllium, or magnesium, and wherein the second metal comprises copper, silver, palladium, platinum, or gold.

13. The sensor of claim 6, further comprising:
a second housing at least partially defining a second chamber containing a second electrolyte, the second housing comprising a gas permeable material, the second housing longitudinally spaced from the first housing;
a third electrode in the second chamber, the third electrode comprising sides and an end, the sides of the third electrode surrounded by a second insulating layer, the end of the third electrode in contact with the second electrolyte, the end of the second electrode not in contact with the second housing, the third electrode comprising a third metal; and
a fourth electrode in the second chamber and substantially parallel to the third electrode, the fourth electrode comprising a fourth metal, wherein a potential difference between the third metal and the fourth metal is at least about 2.5 volts.

14. The sensor of claim 13, wherein the third metal is magnesium, and wherein the fourth metal is copper, silver, palladium, platinum, or gold.

15. The sensor of claim 13, further comprising a third housing at least partially in the second housing, the third housing comprising sides and an end comprising a first frit, the sides of the third housing being gas impermeable, the sides and the end of the third housing at least partially defining a third chamber containing a third electrolyte, wherein the fourth electrode is in the third chamber.

16. The sensor of claim 15, wherein the sides of the third housing comprise polyimide or glass.

17. The sensor of claim 15, further comprising a fourth housing at least partially defining a fourth chamber containing a fourth electrolyte, the first housing at least partially in the fourth chamber.

18. The sensor of claim 17, further comprising a fifth housing at least partially in the first chamber, the fifth housing comprising sides and an end comprising a second frit, the sides of the fifth housing being gas impermeable, the sides and the end of the fifth housing at least partially defining a fifth chamber containing a fifth electrolyte, wherein the second electrode is in the fifth chamber and is in contact with the fifth electrolyte.

19. An implantable probe comprising the sensor of claim 6.

20. A blood gas concentration sensor comprising:
a first housing at least partially defining a first chamber containing a first electrolyte, the first housing comprising a gas permeable material;
a first wire comprising an exposed end comprising a first metal in contact with the first electrolyte; and
a second wire comprising a second metal, wherein a potential difference between the first metal and the second metal is at least about 0.5 volts.
